(19) United States
(12) Patent Application Publication
(10) Pub. No.: US 2013/0165439 A1
(43) Pub. Date: Jun. 27, 2013

(54) CHEMICAL COMPOUNDS

(71) Applicants: Darryl MCCONNELL, Vienna (AT); Lars van der VEEN, Alsbach-Haehnlein (DE); Tobias WUNBERG, Hinterbruehl (AT)

(72) Inventors: Darryl MCCONNELL, Vienna (AT); Lars van der VEEN, Alsbach-Haehnlein (DE); Tobias WUNBERG, Hinterbruehl (AT)

(73) Assignee: Boehringer Ingelheim International GmbH, Ingelheim am Rhein (DE)

(21) Appl. No.: 13/775,601

(22) Filed: Feb. 25, 2013

Related U.S. Application Data
(63) Continuation of application No. 12/665,822, filed on Apr. 13, 2010.

(30) Foreign Application Priority Data
Jun. 25, 2007 (EP) 07110942.5

(51) Int. Cl. A61K 31/5377; A61K 45/06; A61K 31/496; C07D 471/04; A61K 31/437

(52) U.S. Cl. CPC A61K 31/5377; C07D 471/04; A61K 31/437; A61K 31/496; A61K 45/06
USPC 514/234.2; 546/119; 514/303; 544/127; 544/362; 514/253.04

(57) ABSTRACT

The present invention encompasses compounds of general formula (I)

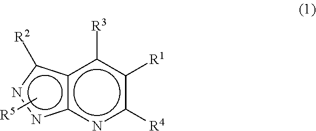

wherein the groups R¹ to R⁵ are defined as in claim 1, which are suitable for treating diseases characterised by excessive or abnormal cell proliferation, and their use for preparing a medicament having the above-mentioned properties.

The present invention relates to new heterocyclic compounds of general formula (I)

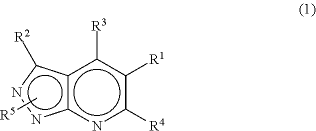

wherein the groups R¹ to R⁵ have the meanings given in the claims and specification, the isomers and salts thereof as well as the use thereof as medicaments.

### BACKGROUND TO THE INVENTION

WO2006/130673 describes pyrazolopyridines which are substituted in 3-position by a benzimidazolyl-group. WO2004/076450 discloses pyrazolopyridine derivatives as p38 kinase inhibitors.

The aim of the present invention is to indicate new active substances which can be used for the prevention and/or treatment of diseases characterised by excessive or abnormal cell proliferation.

### DETAILED DESCRIPTION OF THE INVENTION

Surprisingly, it has been found that compounds of general formula (I), wherein groups R¹ to R⁵ have the meanings given hereinafter, act as inhibitors of specific signal transduction enzymes. Thus the compounds according to the invention may be used for example for the treatment of diseases connected with the activity of specific signal transduction enzymes and characterised by excessive or abnormal cell proliferation.

The present invention therefore relates to compounds of general formula (I)

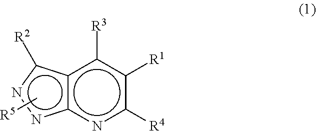

wherein

R¹ is selected from the group consisting of C₁₋₆alkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl, all the above-mentioned groups optionally being substituted by one or more identical or different R⁶; or

R¹ is selected from the group consisting of, —OR^{c}, C₁₋₃haloalkyloxy, —OCF₃, —SR^{c}, —NR^{c}R^{c}, —ONR^{c}R^{c}, —N(OR^{c})R^{c}, —N(R^{g})NR^{c}R^{c}, halogen, —CF₃, —CN, —NC, —OCN, —SCN, —NO, —NO₂, ═N₂, —N₃, —S(O)R^{c}, —S(O)OR^{c}, —S(O)₂R^{c}, —S(O)₂OR^{c}, —S(O)NR^{c}R^{c}, —S(O)₂NR^{c}R^{c}, —OS(O)R^{c}, —OS(O)₂R^{c}, —OS(O)₂OR^{c}, —OS(O)NR^{c}R^{c}, —OS(O)₂NR^{c}R^{c}, —C(O)R^{c}, —C(O)OR^{c}, —C(O)SR^{c}, —C(O)NR^{c}R^{c}, —C(O)N(R^{g})NR^{c}R^{c}, —C(O)N(R^{g})OR^{c}, —C(NR^{g})NR^{c}R^{c}, C(NOH)R^{c}, —C(NOH)NR^{c}R^{c}, —OC(O)R^{c}, —OC(O)OR^{c}, —OC(O)SR^{c}, —OC(O)NR^{c}R^{c}, —OC(NR^{g})NR^{c}R^{c}, —SC(O)R^{c}, —SC(O)OR^{c}, —SC(O)NR^{c}R^{c}, —SC(NR^{g})NR^{c}R^{c}, —N(R^{g})C(O)R^{c}, —N[C(O)R^{c}]₂, —N(OR^{g})C(O)R^{c}, —N(R^{g})C(NR^{g})R^{c}, —N(R^{g})N(R^{g})C(O)R^{c}, —N[C(O)R^{c}]NR^{c}R^{c}, —N(R^{g})C(S)R^{c}, —N(R^{g})S(O)R^{c}, —N(R^{g})S(O)OR^{c}, —N(R^{g})S(O)₂R^{c}, —N[S(O)₂R^{c}]₂, —N(R^{g})S(O)₂OR^{c}, —N(R^{g})S(O)₂NR^{c}R^{c}, —N(R^{g})[S(O)₂]₁R^{c}, —N(R^{g})C(O)OR^{c}, —N(R^{g})C(O)SR^{c}, —N(R^{g})C(O)NR^{c}R^{c}, —N(R^{g})C(O)NR^{g}NR^{c}R^{c}, —N(R^{g})N(R^{g})C(O)NR^{c}R^{c}, —N(R^{g})C(S)NR^{c}R^{c}, —[N(R^{g})C(O)]₂R^{c}, —N(R^{g})[C(O)]₂R^{c}, —N{[C(O)]₂R^{c}}₂, —N(R^{g})[C(O)]₂OR^{c}, —N(R^{g})[C(O)]₂NR^{c}R^{c}, —N{[C(O)]₂OR^{c}}₂, —N{[C(O)]₂NR^{c}R^{c}}₂, —[N(R^{g})C(O)]₂OR^{c}, —N(R^{g})C(NR^{g})OR^{c}, —N(R^{g})C(NOH)R^{c}, —N(R^{g})C(NR^{g})SR^{c} and —N(R^{g})C(NR^{g})NR^{c}R^{c}, and —N═C(R^{g})NR^{c}R^{c}; and

R² denotes a group, optionally substituted by one or more R⁶, selected from among C₃₋₁₀cycloalkyl, 3-8-membered heterocycloalkyl, C₆₋₁₅aryl and 5-12-membered Heteroaryl; and wherein R² is not benzimidazolyl;

R³ and R⁴ independently from each other denotes hydrogen, R^{a} or R^{b},

R⁵ is selected from the group consisting of C₁₋₆alkyl, C₃₋₁₀cycloalkyl, C₄₋₁₆cycloalkylalkyl, C₇₋₁₆arylalkyl, 6-18 membered heteroarylalkyl, 3-14 membered heterocycloalkyl and 4-14 membered heterocycloalkylalkyl, all the above-mentioned groups optionally being substituted by one or more identical or different R^{f}, and R⁵ can be placed on any of the 2 N of the pyrazole ring; and

each R⁶ denotes a group selected from among R^{a}, R^{b} and R^{a} substituted by one or more identical or different R^{c} and/or R^{b};

each R^{a} independently of one another denotes hydrogen or a group optionally substituted by one or more identical or different R^{b} and/or R^{c}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl,

each R^{b} denotes a suitable group and is selected independently of one another from among ═O, —OR^{c}, C₁₋₃haloalkyloxy, —OCF₃, ═S, —SR^{c}, ═NR^{c}, ═NOR^{c}, ═NNR^{c}R^{c}, ═NN(R^{g})C(O)NR^{c}R^{c}, —NR^{c}R^{c}, —ONR^{c}R^{c}, —N(OR^{c})R^{c}, —N(R^{g})NR^{c}R^{c}, halogen, —CF₃, —CN, —NC, —OCN, —SCN, —NO, —NO₂, ═N₂, —N₃, —S(O)R^{c}, —S(O)OR^{c}, —S(O)₂R^{c}, —S(O)₂OR^{c}, —S(O)NR^{c}R^{c}, —S(O)₂NR^{c}R^{c}, —OS(O)R^{c}, —OS(O)₂R^{c}, —OS(O)₂OR^{c}, —OS(O)NR^{c}R^{c}, —OS(O)₂NR^{c}R^{c}, —C(O)R^{c}, —C(O)OR^{c}, —C(O)SR^{c}, —C(O)NR^{c}R^{c}, —C(O)N(R^{g})NR^{c}R^{c}, —C(O)N(R^{g})OR^{c}, —C(NR^{g})NR^{c}R^{c}, —C(NOH)R^{c}, —C(NOH)NR^{c}R^{c}, —OC(O)R^{c}, —OC(O)OR^{c}, —OC(O)SR^{c}, —OC(O)NR^{c}R^{c}, —OC(NR^{g})NR^{c}R^{c}, —SC(O)R^{c}, —SC(O)OR^{c}, —SC(O)NR^{c}R^{c}, —SC(NR^{g})NR^{c}R^{c}, —N(R^{g})C(O)R^{c}, —N[C(O)R^{c}]₂, —N(OR^{g})C(O)R^{c}, —N(R^{g})C(NR^{g})R^{c}, —N(R^{g})N(R^{g})C(O)R^{c}, —N[C(O)R^{c}]NR^{c}R^{c}, —N(R^{g})C(S)R^{c}, —N(R^{g})S(O)R^{c}, —N(R^{g})S(O)OR^{c}, —N(R^{g})S(O)₂R^{c}, —N[S(O)₂R^{c}]₂, —N(R^{g})S(O)₂OR^{c}, —N(R^{g})S(O)₂NR^{c}R^{c}, —N(R^{g})[S(O)₂]₂R^{c}, —N(R^{g})C(O)OR^{c}, —N(R^{g})C(O)SR^{c}, —N(R^{g})C(O)NR^{c}R^{c}, —N(R^{g})C(O)NR^{g}NR^{c}R^{c}, —N(R^{g})N(R^{g})C(O)NR^{c}R^{c}, —N(R^{g})C(S)NR^{c}R^{c}, —[N(R^{g})C(O)]₂R^{c}, —N(R^{g})[C(O)]₂R^{c}, —N{[C(O)]₂R^{c}}₂, —N(R^{g})[C(O)]₂OR^{c}, —N(R^{g})[C(O)]₂NR^{c}R^{c}, —N{[C(O)]₂OR^{c}}₂, —N{[C(O)]₂NR^{c}R^{c}}₂, —[N(R^{g})C(O)]₂OR^{c}, —N(R^{g})C(NR^{g})OR^{c}, —N(R^{g})C(NOH)R^{c}, —N(R^{g})C(NR^{g})SR^{c}, —N(R^{g})C(NR^{g})NR^{c}R^{c} and —N═C(R^{g})NR^{c}R^{c} and

each R^{c} independently of one another denotes hydrogen or a group optionally substituted by one or more identical or different R^{d} and/or R^{e}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl, and each R^{d} denotes a suitable group and is selected independently of one another from among ═O, —OR^{e}, C₁₋₃haloalkyloxy, —OCF₃, ═S, —SR^{e}, ═NR^{e}, ═NOR^{e}, ═NNR^{e}R^{e}, ═NN(R^{g})C(O)NR^{e}R^{e}, —NR^{e}R^{e}, —ONR^{e}R^{e}, —N(R^{g})NR^{e}R^{e}, halogen, —CF₃, —CN, —NC, —OCN, —SCN, —NO, —NO₂, ═N₂, —N₃, —S(O)R^{e}, —S(O)OR^{e}, —S(O)₂R^{e}, —S(O)₂OR^{e}, —S(O)NR^{e}R^{e}, —S(O)₂NR^{e}R^{e}, —OS(O)R^{e}, —OS(O)₂R^{e}, —OS(O)₂OR^{e}, —OS(O)NR^{e}R^{e}, —OS(O)₂NR^{e}R^{e}, —C(O)R^{e}, —C(O)OR^{e}, —C(O)SR^{e}, —C(O)NR^{e}R^{e}, —C(O)N(R^{g})NR^{e}R^{e}, —C(O)N(R^{g})OR^{e}, —C(NR^{g})NR^{e}R^{e}, —C(NOH)R^{e}, —C(NOH)NR^{e}R^{e}, —OC(O)R^{e}, —OC(O)OR^{e}, —OC(O)SR^{e}, —OC(O)NR^{e}R^{e}, —OC(NR^{g})NR^{e}R^{e}, —SC(O)R^{e}, —SC(O)OR^{e}, —SC(O)NR^{e}R^{e}, —SC(NR^{g})NR^{e}R^{e}, —N(R^{g})C(O)R^{e}, —N[C(O)R^{e}]₂, —N(OR^{g})C(O)R^{e}, —N(R^{g})C(NR^{g})R^{e}, —N(R^{g})N(R^{g})C(O)R^{e}, —N[C(O)R^{e}]NR^{e}R^{e}, —N(R^{g})C(S)R^{e}, —N(R^{g})S(O)R^{e}, —N(R^{g})S(O)OR^{e}—N(R^{g})S(O)₂R^{e}, —N[S(O)₂R^{e}]₂, —N(R^{g})S(O)₂OR^{e}, —N(R^{g})S(O)₂NR^{e}R^{e}, —N(R^{g})[S(O)₂]₂R^{e}, —N(R^{g})C(O)OR^{e}, —N(R^{g})C(O)SR^{e}, —N(R^{g})C(O)NR^{e}R^{e}, —N(R^{g})C(O)NR^{g}NR^{e}R^{e}, —N(R^{g})N(R^{g})C(O)NR^{e}R^{e}, —N(R^{g})C(S)NR^{e}R^{e}, —[N(R^{g})C(O)]₂R^{e}, —N(R^{g})[C(O)]₂R^{e}, —N{[C(O)]₂R^{e}}₂, —N(R^{g})[C(O)]₂R^{e}, —N(R^{g})[C(O)]₂NR^{e}R^{e}, —N{[C(O)]₂OR^{e}}₂, —N{[C(O)]₂NR^{e}R^{e}}₂, —[N(R^{g})C(O)]₂OR^{e}, —N(R^{g})C(NR^{g})OR^{e}, —N(R^{g})C(NOH)R^{e}, —N(R^{g})C(NR^{g})SR^{e}, —N(R^{g})C(NR^{g})NR^{e}R^{e} and —N═C(R^{g})NR^{e}R^{e}

each R^{e} independently of one another denotes hydrogen or a group optionally substituted by one or more identical or different R^{f} and/or R^{e}, selected from among C₁₋₆alkyl, 2-6 membered heteroalkyl, C₁₋₆haloalkyl, C₃₋₁₀ cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl, and each R^{f} denotes a suitable group and in each case is selected independently of one another from among ═O, —OR^{g}, C₁₋₃haloalkyloxy, —OCF₃, ═S, —SR^{g}, ═NR^{g}, ═NOR^{g}, ═NNR^{g}R^{g}, ═NN(R^{h})C(O)NR^{g}R^{g}, —NR^{g}R^{g}, —ONR^{g}R^{g}, —N(R^{h})NR^{g}R^{g}, halogen, —CF₃, —CN, —NC, —OCN, —SCN, —NO, —NO₂, ═N₂, —N₃, —S(O)R^{g}, —S(O)OR^{g}, —S(O)₂R^{g}, —S(O)₂OR^{g}, —S(O)NR^{g}R^{g}, —S(O)₂NR^{g}R^{g}, —OS(O)R^{g}, —OS(O)₂R^{g}, —OS(O)₂OR^{g}, —OS(O)NR^{g}R^{g}, —OS(O)₂NR^{g}R^{g}, —C(O)R^{g}, —C(O)OR^{g}, —C(O)SR^{g}, —C(O)NR^{g}R^{g}, —C(O)N(R^{h})NR^{g}R^{g}, —C(O)N(R^{h})OR^{g}, —C(NR^{h})NR^{g}R^{g}, —C(NOH)R^{g}, —C(NOH)NR^{g}R^{g}, —OC(O)R^{g}, —OC(O)OR^{g}, —OC(O)SR^{g}, —OC(O)NR^{g}R^{g}, —OC(NR^{h})NR^{g}R^{g}, —SC(O)R^{g}, —SC(O)OR^{g}, —SC(O)NR^{g}R^{g}, —SC(NR^{h})NR^{g}R^{g}, —N(R^{h})C(O)R^{g}, —N[C(O)R^{g}]₂, —N(OR^{h})C(O)R^{g}, —N(R^{h})C(NR^{h})R^{g}, —N(R^{h})N(R^{h})C(O)R^{g}, —N[C(O)R^{g}]NR^{g}R^{g}, —N(R^{h})C(S)R^{g}, —N(R^{h})S(O)R^{g}, —N(R^{h})S(O)OR^{g}, —N(R^{h})S(O)₂R^{g}, —N[S(O)₂R^{g}]₂, —N(R^{h})S(O)₂OR^{g}, —N(R^{h})S(O)₂NR^{g}R^{g}, —N(R^{h})[S(O)₂]₂R^{g}, —N(R^{h})C(O)OR^{g}, —N(R^{h})C(O)SR^{g}, —N(R^{h})C(O)NR^{g}R^{g}, —N(R^{h})C(O)NR^{h}NR^{g}R^{g}, —N(R^{h})N(R^{h})C(O)NR^{g}R^{g}, —N(R^{h})C(S)NR^{g}R^{g}, —[N(R^{h})C(O)]₂R^{g}, —N(R^{h})[C(O)]₂R^{g}, —N{[C(O)]₂R^{g}}₂, —N(R^{h})[C(O)]₂OR^{g}, —N(R^{h})[C(O)]₂NR^{g}R^{g}, —N{[C(O)]₂OR^{g}}₂, —N{[C(O)]₂NR^{g}R^{g}}₂, —[N(R^{h})C(O)]₂OR^{g}, —N(R^{h})C(NR^{h})OR^{g}, —N(R^{h})C(NOH)R^{g}, —N(R^{h})C(NR^{h})SR^{g}, —N(R^{h})C(NR^{h})NR^{g}R^{g}; and —N═C(R^{h})NR^{h}R^{h}; and

each R^{g} independently of one another denotes hydrogen or a group optionally substituted by one or more identical or different R^{h}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl; and

each R^{h} is selected independently of one another from among hydrogen, C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl,

optionally in the form of the prodrugs, the tautomers, the racemates, the enantiomers, the diastereomers, the prodrugs and the mixtures thereof, and optionally the pharmacologically acceptable salts thereof.

One aspect of the invention are compounds of general formular (1), wherein R³ denotes hydrogen.

A further aspect of the invention are compounds of general formular (1), wherein R⁴ denotes hydrogen.

A further aspect of the invention are compounds of general formular (1), wherein R⁵ denotes C₁₋₃alkyl.

A further aspect of the invention are compounds of general formular (1)—or the pharmaceutically active salts thereof—for use as pharmaceutical compositions.

A further aspect of the invention are compounds of general formular (1)—or the pharmaceutically active salts thereof—for preparing a pharmaceutical composition with an antiproliferative activity.

A further aspect of the invention is a pharmaceutical preparations, containing as active substance one or more compounds of general formula (I) or the physiologically acceptable salts thereof optionally in conjunction with conventional excipients and/or carriers.

A further aspect of the invention is the use of a compound of general formula (I) for preparing a pharmaceutical composition for the treatment and/or prevention of cancer, infections, inflammatory and autoimmune diseases.

A further aspect of the invention is a pharmaceutical preparation comprising a compound of general formula (I) and at least one other cytostatic or cytotoxic active substance, different from formula (I), optionally in the form of the tautomers, the racemates, the enantiomers, the diastereomers and the mixtures thereof, and optionally the pharmacologically acceptable acid addition salts thereof.

### DEFINITIONS

As used herein, the following definitions apply, unless stated otherwise:

The use of the prefix C_{x-y}, wherein x and y in each case represent a natural number (x<y), indicates that the chain or ring structure or combination of chain and ring structure specified and mentioned in direct conjunction may consist of a total of at most y and at least x carbon atoms.

Alkyl is made up of the sub-groups saturated hydrocarbon chains and unsaturated hydrocarbon chains, while the latter may be further subdivided into hydrocarbon chains with a double bond (alkenyl) and hydrocarbon chains with a triple bond (alkynyl).

Alkenyl contains at least one double bond, alkynyl contains at least one triple bond. If a hydrocarbon chain were to carry both at least one double bond and also at least one triple bond, by definition it would belong to the alkynyl sub-group. All the sub-groups mentioned above may further be divided into straight-chain (unbranched) and branched. If an alkyl is substituted, the substitution may be mono- or polysubstitution in each case, at all the hydrogen-carrying carbon atoms, independently of one another.

Examples of representatives of individual sub-groups are listed below.

Straight-Chain (Unbranched) or Branched Saturated Hydrocarbon Chains:

methyl; ethyl; n-propyl; isopropyl (1-methylethyl); n-butyl; 1-methylpropyl; isobutyl (2-methylpropyl); sec.-butyl (1-methylpropyl); tert.-butyl (1,1-dimethylethyl); n-pentyl; 1-methylbutyl; 1-ethylpropyl; isopentyl (3-methylbutyl); neopentyl (2,2-dimethyl-propyl); n-hexyl; 2,3-dimethylbutyl; 2,2-dimethylbutyl; 3,3-dimethylbutyl; 2-methyl-pentyl; 3-methylpentyl; n-heptyl; 2-methylhexyl; 3-methylhexyl; 2,2-dimethylpentyl; 2,3-dimethylpentyl; 2,4-dimethylpentyl; 3,3-dimethylpentyl; 2,2,3-trimethylbutyl; 3-ethylpentyl; n-octyl; n-nonyl; n-decyl etc.

Straight-Chain (Unbranched) or Branched Alkenyl:

vinyl (ethenyl); prop-1-enyl; allyl (prop-2-enyl); isopropenyl; but-1-enyl; but-2-enyl; but-3-enyl; 2-methyl-prop-2-enyl; 2-methyl-prop-1-enyl; 1-methyl-prop-2-enyl; 1-methyl-prop-1-enyl; 1-methylidenepropyl; pent-1-enyl; pent-2-enyl; pent-3-enyl; pent-4-enyl; 3-methyl-but-3-enyl; 3-methyl-but-2-enyl; 3-methyl-but-1-enyl; hex-1-enyl; hex-2-enyl; hex-3-enyl; hex-4-enyl; hex-5-enyl; 2,3-dimethyl-but-3-enyl; 2,3-dimethyl-but-2-enyl; 2-methylidene-3-methylbutyl; 2,3-dimethyl-but-1-enyl; hexa-1,3-dienyl; hexa-1,4-dienyl; penta-1,4-dienyl; penta-1,3-dienyl; buta-1,3-dienyl; 2,3-dimethylbuta-1,3-diene etc.

Straight-Chain (Unbranched) or Branched Alkynyl:

ethynyl; prop-1-ynyl; prop-2-ynyl; but-1-ynyl; but-2-ynyl; but-3-ynyl; 1-methyl-prop-2-ynyl etc.

By the terms propyl, butyl, pentyl, hexyl, heptyl, octyl, nonyl, decyl etc. without any further definition are meant saturated hydrocarbon groups with the corresponding number of carbon atoms, all the isomeric forms being included.

By the terms propenyl, butenyl, pentenyl, hexenyl, heptenyl, octenyl, nonenyl, decenyl etc. without any further definition are meant unsaturated hydrocarbon groups with the corresponding number of carbon atoms and a double bond, all the isomeric forms, i.e. (Z)/(E) isomers, being included where applicable.

By the terms butadienyl, pentadienyl, hexadienyl, heptadienyl, octadienyl, nonadienyl, decadienyl etc. without any further definition are meant unsaturated hydrocarbon groups with the corresponding number of carbon atoms and two double bonds, all the isomeric forms, i.e. (Z)/(E) isomers, being included where applicable.

By the terms propynyl, butynyl, pentynyl, hexynyl, heptynyl, octynyl, nonynyl, decynyl etc. without any further definition are meant unsaturated hydrocarbon groups with the corresponding number of carbon atoms and a triple bond, all the isomeric forms being included.

By the term heteroalkyl are meant groups which can be derived from the alkyl as defined above in its broadest sense if, in the hydrocarbon chains, one or more of the groups —CH₃ are replaced independently of one another by the groups —OH, —SH or —NH₂, one or more of the groups —CH₂— are replaced independently of one another by the groups —O—, —S— or —NH—, one or more of the groups

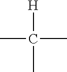

are replaced by the group

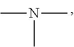

one or more of the groups ═CH— are replaced by the group ═N—, one or more of the groups ═CH₂ are replaced by the group ═NH or one or more of the groups ≡CH are replaced by the group ≡N, while overall there may only be a maximum of three heteroatoms in a heteroalkyl, there must be at least one carbon atom between two oxygen atoms and between two sulphur atoms or between one oxygen and one sulphur atom and the group as a whole must be chemically stable.

It is immediately apparent from the indirect definition/derivation from alkyl that heteroalkyl is made up of the sub-groups saturated hydrocarbon chains with heteroatom(s), heteroalkenyl and heteroalkynyl, and one further subdivision may be carried out into straight-chain (unbranched) and branched. If a heteroalkyl is substituted, the substitution may be mono- or polysubstitution in each case, at all the hydrogen-carrying oxygen, sulphur, nitrogen and/or carbon atoms, independently of one another. Heteroalkyl itself may be linked to the molecule as a substituent both via a carbon atom and via a heteroatom.

Typical examples are listed below:

dimethylaminomethyl; dimethylaminoethyl (1-dimethylaminoethyl; 2-dimethyl-aminoethyl); dimethylaminopropyl (1-dimethylaminopropyl, 2-dimethylaminopropyl, 3-dimethylaminopropyl); diethylaminomethyl; diethylaminoethyl (1-diethylaminoethyl, diethylaminoethyl); diethylaminopropyl (1-diethylaminopropyl, 2-diethylamino-propyl, 3-diethylaminopropyl); diisopropylaminoethyl (1-diisopropylaminoethyl, 2-di-isopropylaminoethyl); bis-2-methoxyethylamino; [2-(dimethylamino-ethyl)-ethyl-amino]-methyl; 3-[2-(dimethylamino-ethyl)-ethyl-amino]-propyl; hydroxymethyl; 2-hydroxy-ethyl; 3-hydroxypropyl; methoxy; ethoxy; propoxy; methoxymethyl; 2-methoxyethyl etc.

Haloalkyl is derived from alkyl as hereinbefore defined in its broadest sense, when one or more hydrogen atoms of the hydrocarbon chain are replaced independently of one another by halogen atoms, which may be identical or different. It is immediately apparent from the indirect definition/derivation from alkyl that haloalkyl is made up of the sub-groups saturated halohydrocarbon chains, haloalkenyl and haloalkynyl, and further subdivision may be made into straight-chain (unbranched) and branched. If a haloalkyl is substituted, the substitution may be mono- or polysubstitution in each case, at all the hydrogen-carrying carbon atoms, independently of one another.

Typical examples are listed below:

—CF₃; —CHF₂; —CH₂F; —CF₂CF₃; —CHFCF₃; —CH₂CF₃; —CF₂CH₃; —CHFCH₃; —CF₂CF₂CF₃; —CF₂CH₂CH₃; —CF═CF₂; —C≡Cl═CH₂; —CBr═CH₂; —CI═CH₂; —C≡C—CF₃; —CHFCH₂CH₃; —CHFCH₂CF₃ etc.

Halogen denotes fluorine, chlorine, bromine and/or iodine atoms.

Cycloalkyl is made up of the sub-groups monocyclic hydrocarbon rings, bicyclic hydrocarbon rings and spirohydrocarbon rings, while each sub-group may be further subdivided into saturated and unsaturated (cycloalkenyl). The term unsaturated means that in the ring system in question there is at least one double bond, but no aromatic system is formed. In bicyclic hydrocarbon rings two rings are linked such that they have at least two carbon atoms in common. In spirohydrocarbon rings one carbon atom (spiroatom) is shared by two rings. If a cycloalkyl is substituted, the substitution may be mono- or polysubstitution in each case, at all the hydrogen-carrying carbon atoms, independently of one another. Cycloalkyl itself may be linked to the molecule as substituent via any suitable position of the ring system.

Typical examples of individual sub-groups are listed below.

Monocyclic Hydrocarbon Rings, Saturated:

cyclopropyl; cyclobutyl; cyclopentyl; cyclohexyl; cycloheptyl etc.

Monocyclic Hydrocarbon Rings Unsaturated:

cycloprop-1-enyl; cycloprop-2-enyl; cyclobut-1-enyl; cyclobut-2-enyl; cyclopent-1-enyl; cyclopent-2-enyl; cyclopent-3-enyl; cyclohex-1-enyl; cyclohex-2-enyl; cyclohex-3-enyl; cyclohept-1-enyl; cyclohept-2-enyl; cyclohept-3-enyl; cyclohept-4-enyl; cyclobuta-1,3-dienyl; cyclopenta-1,4-dienyl; cyclopenta-1,3-dienyl; cyclopenta-2,4-dienyl; cyclohexa-1,3-dienyl; cyclohexa-1,5-dienyl; cyclohexa-2,4-dienyl; cyclohexa-1,4-dienyl; cyclohexa-2,5-dienyl etc.

Bicyclic Hydrocarbon Rings (Saturated and Unsaturated):

bicyclo[2.2.0]hexyl; bicyclo[3.2.0]heptyl; bicyclo[3.2.1]octyl; bicyclo[2.2.2]octyl; bicyclo[4.3.0]nonyl (octahydroindenyl); bicyclo[4.4.0]decyl (decahydronaphthalene); bicyclo[2,2,1]heptyl (norbornyl); (bicyclo[2.2.1]hepta-2,5-dienyl (norborna-2,5-dienyl); bicyclo[2,2,1]hept-2-enyl (norbornenyl); bicyclo[4.1.0]heptyl (norcaranyl); bicyclo-[3.1.1]heptyl (pinanyl) etc.

Spirohydrocarbon Rings (Saturated and Unsaturated):

spiro[2.5]octyl, spiro[3.3]heptyl, spiro[4.5]dec-2-enyl etc.

Cycloalkylalkyl denotes the combination of the above-defined groups alkyl and cycloalkyl, in each case in their broadest sense. The alkyl group as substituent is directly linked to the molecule and is in turn substituted by a cycloalkyl group. The alkyl and cycloalkyl may be linked in both groups via any carbon atoms suitable for this purpose. The respective sub-groups of alkyl and cycloalkyl are also included in the combination of the two groups.

Aryl denotes mono-, bi- or tricyclic carbon rings with at least one aromatic ring. If an aryl is substituted, the substitution may be mono- or polysubstitution in each case, at all the hydrogen-carrying carbon atoms, independently of one another. Aryl itself may be linked to the molecule as substituent via any suitable position of the ring system.

Typical examples are listed below.

phenyl; naphthyl; indanyl (2,3-dihydroindenyl); 1,2,3,4-tetrahydronaphthyl; fluorenyl etc.

Arylalkyl denotes the combination of the groups alkyl and aryl as hereinbefore defined, in each case in their broadest sense. The alkyl group as substituent is directly linked to the molecule and is in turn substituted by an aryl group. The alkyl and aryl may be linked in both groups via any carbon atoms suitable for this purpose. The respective sub-groups of alkyl and aryl are also included in the combination of the two groups.

Typical examples are listed below:

benzyl; 1-phenylethyl; 2-phenylethyl; phenylvinyl; phenylallyl etc.

Heteroaryl denotes monocyclic aromatic rings or polycyclic rings with at least one aromatic ring, which, compared with corresponding aryl or cycloalkyl, contain instead of one or more carbon atoms one or more identical or different heteroatoms, selected independently of one another from among nitrogen, sulphur and oxygen, while the resulting group must be chemically stable. If a heteroaryl is substituted, the substitution may be mono- or polysubstitution in each case, at all the hydrogen-carrying carbon and/or nitrogen atoms, independently of one another. Heteroaryl itself as substituent may be linked to the molecule via any suitable position of the ring system, both carbon and nitrogen.

Typical examples are listed below.

Monocyclic Heteroaryls:

furyl; thienyl; pyrrolyl; oxazolyl; thiazolyl; isoxazolyl; isothiazolyl; pyrazolyl; imidazolyl; triazolyl; tetrazolyl; oxadiazolyl; thiadiazolyl; pyridyl; pyrimidyl; pyridazinyl; pyrazinyl; triazinyl; pyridyl-N-oxide; pyrrolyl-N-oxide; pyrimidinyl-N-oxide; pyridazinyl-N-oxide; pyrazinyl-N-oxide; imidazolyl-N-oxide; isoxazolyl-N-oxide; oxazolyl-N-oxide; thiazolyl-N-oxide; oxadiazolyl-N-oxide; thiadiazolyl-N-oxide; triazolyl-N-oxide; tetrazolyl-N-oxide etc.

Polycyclic Heteroaryls:

indolyl; isoindolyl; benzofuryl; benzothienyl; benzoxazolyl; benzothiazolyl; benzisoxazolyl; benzisothiazolyl; benzimidazolyl; indazolyl; isoquinolinyl; quinolinyl; quinoxalinyl; cinnolinyl; phthalazinyl; quinazolinyl; benzotriazinyl; indolizinyl; oxazolopyridyl; imidazopyridyl; naphthyridinyl; indolinyl; isochromanyl; chromanyl; tetrahydroisoquinolinyl; isoindolinyl; isobenzotetrahydrofuryl; isobenzotetrahydrothienyl; isobenzothienyl; benzoxazolyl; pyridopyridyl; benzotetrahydrofuryl; benzotetrahydro-thienyl; purinyl; benzodioxolyl; phenoxazinyl; phenothiazinyl; pteridinyl; benzothiazolyl; imidazopyridyl; imidazothiazolyl; dihydrobenzisoxazinyl; benzisoxazinyl; benzoxazinyl; dihydrobenzisothiazinyl; benzopyranyl; benzothiopyranyl; cumarinyl; isocumarinyl; chromonyl; chromanonyl; tetrahydroquinolinyl; dihydroquinolinyl; dihydroquinolinonyl; dihydroisoquinolinonyl; dihydrocumarinyl; dihydroisocumarinyl; isoindolinonyl; benzodioxanyl; benzoxazolinonyl; quinolinyl-N-oxide; indolyl-N-oxide; indolinyl-N-oxide; isoquinolyl-N-oxide; quinazolinyl-N-oxide; quinoxalinyl-N-oxide; phthalazinyl-N-oxide; indolizinyl-N-oxide; indazolyl-N-oxide; benzothiazolyl-N-oxide; benzimidazolyl-N-oxide; benzo-thiopyranyl-5-oxide and benzothiopyranyl-S,S-dioxide etc.

Heteroarylalkyl denotes the combination of the alkyl and heteroaryl groups defined hereinbefore, in each case in their broadest sense. The alkyl group as substituent is directly linked to the molecule and is in turn substituted by a heteroaryl group. The linking of the alkyl and heteroaryl may be achieved on the alkyl side via any carbon atoms suitable for this purpose and on the heteroaryl side by any carbon or nitrogen atoms suitable for this purpose. The respective sub-groups of alkyl and heteroaryl are also included in the combination of the two groups.

By the term heterocycloalkyl are meant groups which are derived from the cycloalkyl as hereinbefore defined if in the hydrocarbon rings one or more of the groups —CH₂— are replaced independently of one another by the groups —O—, —S— or —NH— or one or more of the groups ═CH— are replaced by the group ═N—, while not more than five heteroatoms may be present in total, there must be at least one carbon atom between two oxygen atoms and between two sulphur atoms or between one oxygen and one sulphur atom and the group as a whole must be chemically stable. Heteroatoms may simultaneously be present in all the possible oxidation stages (sulphur→sulphoxide-SO—, sulphone-SO₂—; nitrogen→N-oxide). It is immediately apparent from the indirect definition/derivation from cycloalkyl that heterocycloalkyl is made up of the sub-groups monocyclic hetero-rings, bicyclic hetero-rings and spirohetero-rings, while each sub-group can also be further subdivided into saturated and unsaturated (heterocycloalkenyl). The term unsaturated means that in the ring system in question there is at least one double bond, but no aromatic system is formed. In bicyclic hetero-rings two rings are linked such that they have at least two atoms in common. In spirohetero-rings one carbon atom (spiroatom) is shared by two rings. If a heterocycloalkyl is substituted, the substitution may be mono- or polysubstitution in each case, at all the hydrogen-carrying carbon and/or nitrogen atoms, independently of one another. Heterocycloalkyl itself as substituent may be linked to the molecule via any suitable position of the ring system.

Typical examples of individual sub-groups are listed below.

Monocyclic Heterorings (Saturated and Unsaturated):

tetrahydrofuryl; pyrrolidinyl; pyrrolinyl; imidazolidinyl; thiazolidinyl; imidazolinyl; pyrazolidinyl; pyrazolinyl; piperidinyl; piperazinyl; oxiranyl; aziridinyl; azetidinyl; 1,4-dioxanyl; azepanyl; diazepanyl; morpholinyl; thiomorpholinyl; homomorpholinyl; homopiperidinyl; homopiperazinyl; homothiomorpholinyl; thiomorpholinyl-5-oxide; thiomorpholinyl-S,S-dioxide; 1,3-dioxolanyl; tetrahydropyranyl; tetrahydrothiopyranyl; [1,4]-oxazepanyl; tetrahydrothienyl; homothiomorpholinyl-S,S-dioxide; oxazolidinonyl; dihydropyrazolyl; dihydropyrrolyl; dihydropyrazinyl; dihydropyridyl; dihydro-pyrimidinyl; dihydrofuryl; dihydropyranyl; tetrahydrothienyl-5-oxide; tetrahydrothienyl-S,S-dioxide; homothiomorpholinyl-5-oxide; 2,3-dihydroazet; 2H-pyrrolyl; 4H-pyranyl; 1,4-dihydropyridinyl etc.

Bicyclic Heterorings (Saturated and Unsaturated):

8-azabicyclo[3.2.1]octyl; 8-azabicyclo[5.1.0]octyl; 2-oxa-5-azabicyclo[2.2.1]heptyl; 8-oxa-3-aza-bicyclo[3.2.1]octyl; 3.8-diaza-bicyclo[3.2.1]octyl; 2.5-diaza-bicyclo-[2.2.1]heptyl; 1-aza-bicyclo[2.2.2]octyl; 3.8-diaza-bicyclo[3.2.1]octyl; 3.9-diaza-bicyclo[4.2.1]nonyl; 2.6-diaza-bicyclo[3.2.2]nonyl etc.

Spiro-Heterorings (Saturated and Unsaturated):

1,4-dioxa-spiro[4.5]decyl; 1-oxa-3.8-diaza-spiro[4.5]decyl; and 2,6-diaza-spiro[3.3]heptyl; 2,7-diaza-spiro[4.4]nonyl; 2,6-diaza-spiro[3.4]octyl; 3,9-diaza-spiro[5.5]undecyl; 2,8-diaza-spiro[4.5]decyl etc.

Heterocycloalkylalkyl denotes the combination of the alkyl and heterocycloalkyl groups defined hereinbefore, in each case in their broadest sense. The alkyl group as substituent is directly linked to the molecule and is in turn substituted by a heterocycloalkyl group. The linking of the alkyl and heterocycloalkyl may be achieved on the alkyl side via any carbon atoms suitable for this purpose and on the heterocycloalkyl side by any carbon or nitrogen atoms suitable for this purpose. The respective sub-groups of alkyl and heterocycloalkyl are also included in the combination of the two groups.

The term “substituted” indicates that a hydrogen atom which is bound directly to the atom in question is replaced by another atom or another group of atoms. Bivalent substituents such as for example ═O, ═S, ═NR, ═NOR, ═NNRR, ═NN(R)C(O)NRR, ═N₂ or the like can only be substituents at carbon atoms. They require exchanging for two geminal hydrogen atoms, i.e. hydrogen atoms which are bound to the same carbon atom saturated before the substitution. Substitution by a bivalent substituent is therefore only possible at the groups —CH₃ and —CH₂—, not at the groups

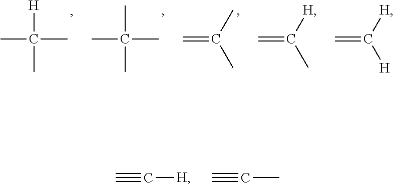

and not at aromatic carbon atoms.

Additionally, by the term “suitable substituent/suitable group” is meant a substituent which on the one hand is suitable on account of its valency and on the other hand leads to a system with chemical stability.

Features and advantages of the present invention will become apparent from the following detailed Examples, which illustrate the basics of the invention by way of example, without limiting its scope.

### Preparation of the Compounds According to the Invention

General

All the reactions are carried out—unless stated otherwise—in commercially obtainable apparatus using methods conventionally used in chemical laboratories.

Air- and/or moisture-sensitive starting materials are stored under protective gas and corresponding reactions and manipulations using them are carried out under protective gas (nitrogen or argon).

Chromatography

Method A:

HPLC: Spectra SYSTEM AS1000; MS: Gilson liquid handler, Finnigan, APCI(+);

Mode: Scan pos 120-730.

Column: Develosil; Part No. 1708689, C18, 5 μm; 4.6 mm×50 mm column

Mobile Phase: A: H₂O desalted with 0.1% TFA
B: Acetonitril HPLC grade

Wavelengths: 220 nm and 254 nm

Injection: 10-20 L standard injection

Flow rate: 1.5 mL/min

Temperature: 25 C°

Gradient: 0.0-0.5 min 5% B
0.5-5.0 min 5%->100% B
5.0-6.2 min 100%->100% B

Preparative HPLC normal phase: Gilson liquid handler, Finnigan,

(APCI (+); Mode: Scan pos 120-730.

Säule: Macherey-Nagel VP100/21 Nucleosil 50-100;

Part No. 715776.210, C18, 10 m; 21 mm×100 mm column

### Synthesis of Reagents

All reagents used in the synthesis of the listed examples are either commercially available or accessible via known or analogous literature synthesis procedures.

### Synthesis of Examples

All examples listed can be synthesized via the outlined synthesis routes A1, A2 and B or using known or analogous literature synthesis procedures.

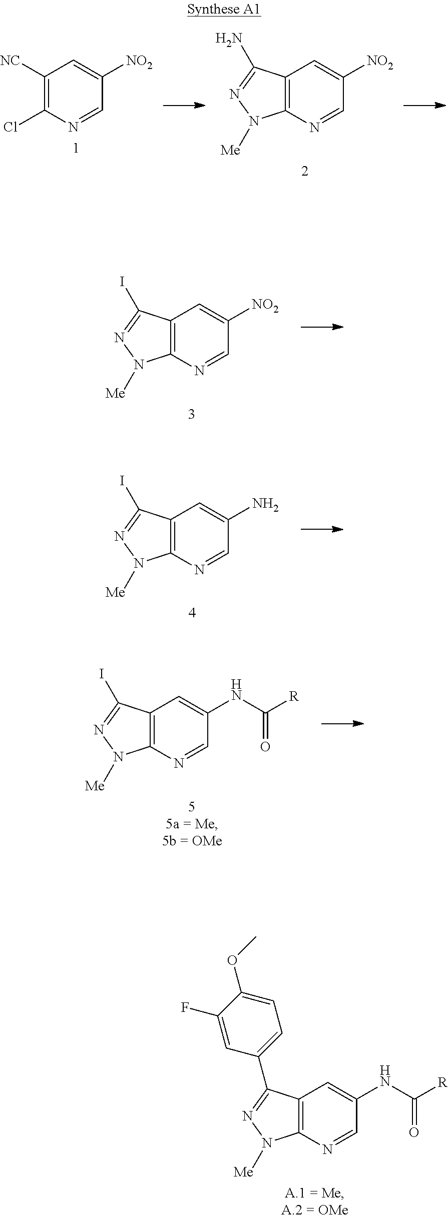

### 1-Methyl-5-nitro-1H-pyrazolo[3,4-b]pyridin-3-amine (2)

Methyl hydrazine (21.5 mL, 0.41 mol, 3 eq.) is added to a suspension of 1 (25 g, 0.136 mol) and cesium carbonate (66.58 g, 0.2 mol, 1.5 eq.) in dimethylformamide (250 mL) under an atmosphere of nitrogen at −12° C. and the reaction mixture is stirred for min. The iced bath is removed and the reaction mixture is stirred for a further 16 h at room temperature. The reaction mixture is then poured into iced water (250 mL). Dichloromethane (350 mL) is added and the mixture is stirred till a fine precipitate formed. The solid is filtered, washed with water (3×250 mL), then diethyl ether (3×250 mL) and dried under vacuum to afford 18.67 g (71%) of the title compound 2 as a red solid. The organic and aqueous liquors are extracted with isopropyl alcohol:chloroform (1:1, 3×300 mL), the organic liquors are combined, dried, filtered and concentrated to afford 7.05 g (27%) of the title compound 2 as solid. C₇H₇N₅O₂ (193.1): MS-APCI: 193.9 ([M+H]⁺). HPLC (Method A) R_{T} in min (purity)=2.90 (99).

### 3-Iodo-1-methyl-5-nitro-1H-pyrazolo[3,4-b]pyridine (3)

Isoamylnitrite (18.8 mL, 0.14 mol, 20 eq.) is added to a suspension of 2 (1.35 g, 7.0 mmol) in diiodomethane (38 mL) under an atmosphere of nitrogen at room temperature. The reaction mixture is stirred for 30 min then hydroiodic acid (135 L, cat.) is added dropwise and the reaction mixture is stirred for a further 1.5 h. The reaction mixture is poured into 12.5% ammonium hydroxide solution in water (300 mL) and stirred for 15 min. The whole is extracted with dichloromethane (3×200 mL), the organic liquors are combined, dried, filtered and concentrated to afford an orange liquid. The liquid is co-evaporated with a mixture of methanol:acetone (10:1, 4×150 mL) to afford a solid. The crude material is purified by flash column chromatography over silica gel, eluting with hexanes:ethyl acetate:methanol (25:1:0 to 7.5:1:0.25) to afford 731 mg (34%) of the title compound 3. A further fraction of 3 614 mg (29%) is isolated by flushing the column with ethyl acetate:methanol (1:0.1). C₇H₅IN₄O₂ (304.0): MS-APCI: 304.8 ([M+H]⁺). HPLC (Method A) R_{T} in min (purity)=4.42 (93).

### 3-Iodo-1-methyl-1H-ppyrazolo[3,4-b]pyridin-5-amine (4)

A reaction vessel is evacuated and purged with nitrogen (×3) before platinum on carbon 5% (12.88 g) is added to a suspension of 3 (8.99 g, 29.6 mmol), triethylamine (412 μL, 2.96 mmol, 0.1 eq.), a 4% solution of thiophene in diisopropylether (12.88 mL) and vanadium(V) oxide (1.61 g, 8.9 mmol, 0.3 eq.) in a mixture of tetrahydrofuran:dimethylform-amide (494 mL, 1:1). The reaction vessel is then evacuated and purged with hydrogen gas (×3) and the reaction mixture is stirred under an atmosphere of hydrogen for 2 h. The reaction mixture is filtered through a pad of Celite® and silica and the cake is washed with ethyl acetate (300 mL). The filtrate is co-evaporated with toluene (4×300 mL) and the crude material was purified by flash column chromatography over silica gel, eluting with hexanes:ethyl acetate:methanol (5:1:0.05 to 5:2:0.2) to afford 5.07 g (63%) of the title compound 4. C₇H₇₁N₄ (274.0): MS-APCI: 274.8 ([M+H]⁺). HPLC (Method A) R_{T} in min (purity)=2.08 (95).

### N-(3-Iodo-1-methyl-1H-ppyrazolo[3,4-b]pyridin-5-yl)acetamide (5a)

Acetic anhydride (2.26 mL, 24.43 mmol, 1.5 eq.) is added to a solution of 4 (4.02 g, 14.67 mmol) and pyridine (2.11 mL, 26.1 mmol, 1.6 eq.) in dichloromethane (140 mL) under an atmosphere of nitrogen at 0° C. and the reaction mixture is stirred for 25 min. The ice bath is removed and the reaction mixture was stirred for a further 16 h at room temperature. The precipitate formed is filtered, washed with cold dichloromethane (50 mL), cold diethyl ether (50 mL), water (2×50 mL), cold diethyl ether (2×75 mL) and is then dried under vacuum to afford 4.19 g (96%) of the title compound 5a. C₉H₉₁N₄O (316.1): MS-APCI: 316.8 ([M+H]⁺). HPLC (Method A) R_{T} in min (purity)=3.13 (100).

### Methyl-3-iodo-1-methyl-1H-pyrazolo[3,4-b]pyridin-5-ylcarbamate (5b)

Methyl chloroformate (47 μL, 0.6 mmol, 1.25 eq.) is added to a solution of 4 (134 mg, 0.5 mmol) and pyridine (51 μL, 0.63 mmol, 1.3 eq.) in dichloromethane (7 mL) under an atmosphere of nitrogen at 0° C. and the reaction mixture is stirred for 25 min. The ice bath is removed and the reaction mixture is stirred for a further 6 h at room temperature. The reaction mixture is washed with water (3×10 mL), dried, filtered and concentrated. The crude material is purified by flash column chromatography over silica gel, eluting with hexanes:ethyl acetate (1:1 to 0:1) to afford 96 mg (59%) of the title compound 5b. C₉H₉IN₄O₂ (332.1): MS-APCI: 332.8 ([M+H]⁺). HPLC (Method A) R_{T} in min (purity)=3.64 (100).

### N-(3-(3-Fluoro-4-methoxyphenyl)-1-methyl-1H-pyrazolo[3,4-b]pyridin-5-yl)acetamide (A.1)

Palladium acetate (2.5 mg, 0.011 mmol, 0.05 eq.) is added to a suspension of 5a (70 mg, 0.22 mmol), 3-fluoro-4-methoxyphenyl boronic acid (60 mg, 0.35 mmol, 1.6 eq.), potassium phosphate (94 mg, 0.44 mmol, 2 eq.), (2-biphenyl)dicyclohexyl phosphine (7.8 mg, 0.022 mmol, 0.1 eq.) in a degassed mixture of toluene:water (3.6 mL, 5:1). The reaction mixture is stirred under an atmosphere of nitrogen at 82° C. for 16 h. The reaction is filtered, the solid collected and washed with toluene (5 mL), water (2×5 mL) then toluene (2×5 mL). The remaining crude material is washed with diethyl ether and the filtrate collected and concentrated to afford 53 mg (76%) of the title compound A.1. C₁₆H₁₅FN₄O₂ (314.3): MS-APCI: 314.9 ([M+H]⁺). HPLC (Method A) R_{T} in min (purity)=3.77 (100).

### Methyl 3-(3-fluoro-4-methoxyphenyl)-1-methyl-1H-pyrazolo[3,4-b]pyridin-5-ylcarbamate (A.2)

Palladium acetate (2.8 mg, 0.013 mmol, 0.05 equ.) is added to a suspension of 5b (83 mg, 0.22 mmol), 3-fluoro-4-methoxyphenyl boronic acid (68 mg, 0.4 mmol, 1.6 eq.), potassium phosphate (106 mg, 0.5 mmol, 2 eq.), (2-biphenyl)dicyclohexyl phosphine (8.8 mg, 0.025 mmol, 0.1 eq.) in a degassed mixture of toluene:water (3.6 mL, 5:1). The reaction mixture is stirred under an atmosphere of nitrogen at 85° C. for 5 h. The reaction mixture is then filtered through a pad of silica and the cake is washed with ethyl acetate (30 mL) and the filtrate concentrated. The crude material is purified by flash column chromatography over silica gel, eluting with hexanes:ethyl acetate (1:1 to 0:1) to afford 53 mg (76%) of the title compound A.2. C₁₆H₁₅FN₄O₃ (330.3): MS-APCI: 331.0 ([M+H]⁺). HPLC (Method A) R_{T} in min (purity)=4.27 (98).

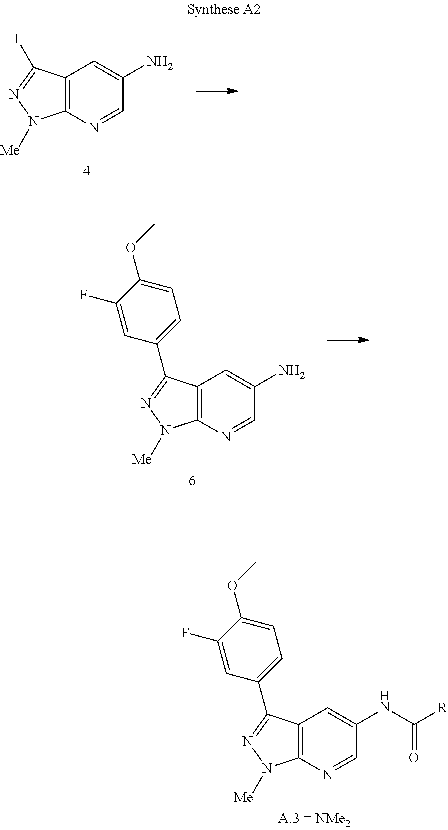

### 3-(3-Fluoro-4-methoxyphenyl)-1-methyl-1H-pyrazolo[3,4-b]pyridin-5-amine (6)

Palladium acetate (36.8 mg, 0.164 mmol, 0.05 eq.) is added to a suspension of 4 (0.99 g, 3.61 mmol), 3-fluoro-4-methoxyphenyl boronic acid (982 mg, 5.77 mmol, 1.6 eq.), potassium phosphate (1.53 g, 7.22 mmol, 2 eq.), (2-biphenyl)dicyclohexyl phosphine (126 mg, 0.36 mmol, 0.1 eq.) in a degassed mixture of toluene:water (36 mL, 5:1). The reaction mixture is stirred under an atmosphere of nitrogen at 85° C. for 2.5 h. The reaction is filtered, the solid collected and washed with toluene (50 mL), water (2×50 mL), toluene (50 mL), and then diethyl ether (2×50 mL). The remaining solid is washed through the sinter with hot diethyl ether (3×50 mL) and the filtrate is evaporated to afford 692 mg (70%) of the title compound 6. C₁₄H₁₃FN₄O (272.2): MS-APCI: 272.9 ([M+H]⁺). HPLC (system A) R_{T} in min (purity)=3.11 (100). A 40 mg sample is purified further by flash column chromatography over silica gel, eluting with ethyl acetate:methanol (1:0 to 1:0.1) to afford 4 0 mg of 6 as a screening sample (removal of residual traces of Pd).

### 3-(3-(3-Fluoro-4-methoxyphenyl)-1-methyl-1H-pyrazolo[3,4-b]pyridine-5-yl)-1,1-dimethylurea (A.3)

Dimethyl carbamyl chloride (51 μL, 0.55 mmol, 1.5 eq.) is added to a solution of 6 (100 mg, 0.37 mmol) and pyridine (48 μL, 0.59 mmol, 1.6 eq.) in dichloromethane (4 mL) under an atmosphere of nitrogen at 0° C. and the reaction mixture is stirred for 20 min. The ice bath is removed and the reaction mixture is stirred for a further 2 h at room temperature then heated at 50° C. for 2 h. The precipitate formed is filtered under vacuum and washed with water, the crude material is then purified by normal phase preparative HPLC (eluting with hexane:ethyl acetate:methanol gradients) to afford 64 mg (51%) of the title compound A.3. C₁₇H₁₈FN₅O₂ (343.3): MS-APCI: 344.0 ([M+H]⁺). HPLC (system A) R_{T} in min (purity)=3.80 (97).

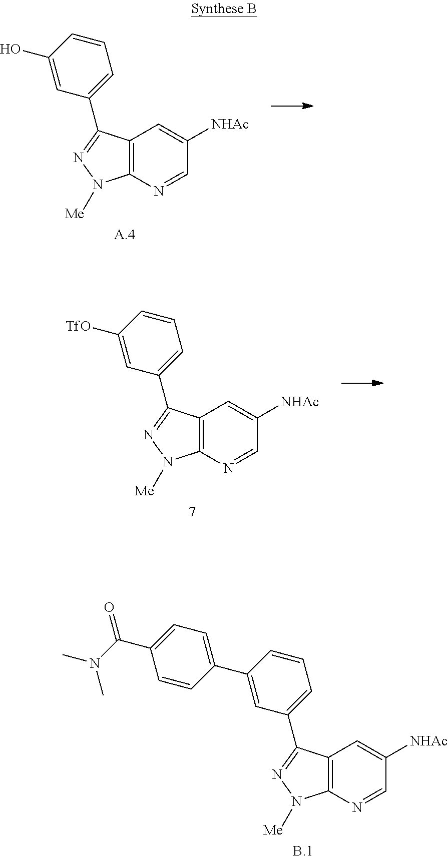 [SMILES: CC(=O)Nc1cnc2c(c1)c(-c1cccc(O)c1)nn2C ; CC(=O)Nc1cnc2c(c1)c(-c1cccc(OS(=O)(=O)C(F)(F)F)c1)nn2C ; CC(=O)Nc1cnc2c(c1)c(-c1cccc(-c3ccc(C(=O)N(C)C)cc3)c1)nn2C]

### N-(3-(3-Hydroxyphenyl)-1-methyl-1H-pyrazolo[3,4-b]pyridine-5-yl)acetamide (A.4)

Palladium acetate (35.5 mg, 0.16 mmol, 0.05 eq.) is added to a suspension of 5a (1.0 g, 3.16 mmol), 3-hydroxyphenyl boronic acid (698 mg, 5.10 mmol, 1.6 eq.), potassium phosphate (1.34 g, 6.33 mmol, 2 eq.), (2-biphenyl)dicyclohexyl phosphine (111 mg, 0.32 mmol, 0.1 eq.) in a degassed mixture of toluene:water (36 mL, 5:1). The reaction mixture is stirred under an atmosphere of nitrogen at 85° C. for 2.5 h. The reaction is filtered, the solid collected and washed with toluene (50 mL), water (2×50 mL), then diethyl ether (2×50 mL). The crude material is purified by flash column chromatography over silica gel, eluting with hexanes:ethyl acetate:methanol (5:5:0.2 to 5:5:1) to afford 755 mg (85%) of the title compound A.4. C₁₅H₁₄N₄O₂ (282.3): MS-APCI: 282.9 ([M+H]⁺). HPLC (Method A) R_{T} in min (purity)=3.11 (100).

### 3-(5-Acetamido-1-methyl-1H-pyrazolo[3,4-b]pyridine-3-yl)phenyltrifluoromethane sulfonate (7)

Triflic anhydride (1.9 mL, 11.6 mmol, 2 eq.) is added to a solution of A.4 (1.65 g, 5.85 mmol) in dichloromethane:pyridine (45 mL, 2:1) under an atmosphere of nitrogen at −15° C. and the reaction mixture is stirred for 20 min. The reaction is allowed to warm to room temperature over ca 1 h then poured on to ice and extracted with dichloromethane (2×30 mL). The organic liquors are combined, dried, filtered and concentrated. The crude material is purified by flash column chromatography over silica gel, eluting with dichloromethane:acetone (10:2 to 8:2) to afford 1.85 g (76%) of the title compound 7. C₁₆H₁₃F₃N₄O₄S (414.3): MS-APCI: 414.9 ([M+H]⁺). HPLC (system A) R_{T} in min (purity)=4.49 (100).

### 3′-(5-Acetamido-1-methyl-1H-pyrazolo[3,4-b]pyridine-3-yl)-N,N-dimethylbiphenyl-4-carboxamide (B.1)

Palladium acetate (3 mg, 0.013 mmol, 0.05 equ.) is added to a suspension of 7 (110 mg, 0.27 mmol), 4-(dimethylcarbamoyl)phenylboronic acid (82 mg, 0.43 mmol, 1.6 eq.), potassium phosphate (115 mg, 0.54 mmol, 2 eq.), (2-biphenyl)dicyclohexyl phosphine (9.3 mg, 0.027 mmol, 0.1 eq.) in a degassed mixture of toluene:water (4.44 mL, 5:1). The reaction mixture is stirred under an atmosphere of nitrogen at 85° C. for 16 h. The layer of toluene is removed and the aqueous layer is washed with toluene (3 mL). The aqueous layer is extracted with dichloromethane (3×5 mL), the organic layers are combined and washed through a pad of MgSO₄ and silica eluting with hot diethyl ether, dichloromethane, ethyl acetate and finally 10% methanol in ethyl acetate to afford 64 mg (58%) of the title compound B.1. C₂₄H₂₃N₅O₂ (413.4): MS-APCI: 414.0 ([M+H]⁺). HPLC (system A) R_{T} in min (purity)=3.80 (98).

### Examples A.1-A.18

Examples A.1-A.18 are prepared according to synthesis A.1 or A.2.

| | | | Mass | HPLC Rt |
|---|---|---|---|---|
| # | Educt | Structure | [M + 1]⁺ | [min] |
| A.1 | 5a | 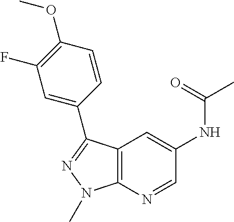 [SMILES: COc1ccc(-c2nn(C)c3ncc(NC(C)=O)cc23)cc1F] | 314.9 | 3.77 |
| A.2 | 5b | 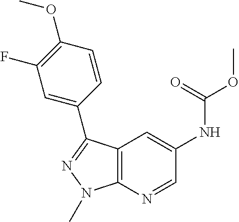 [SMILES: COC(=O)Nc1cnc2c(c1)c(-c1ccc(OC)c(F)c1)nn2C] | 331.0 | 4.27 |
| A.3 | 6 | 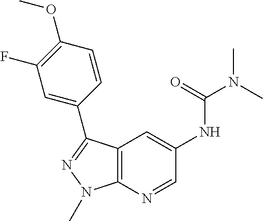 [SMILES: COc1ccc(-c2nn(C)c3ncc(NC(=O)N(C)C)cc23)cc1F] | | |
| A.4 | 5a | 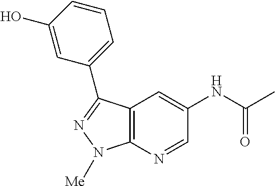 [SMILES: CC(=O)Nc1cnc2c(c1)c(-c1cccc(O)c1)nn2C] | 282.9 | 3.11 |
| A.5 | A.1 | 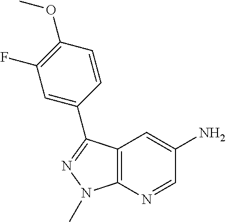 [SMILES: COc1ccc(-c2nn(C)c3ncc(N)cc23)cc1F] | | |
| A.6 | A.5 | 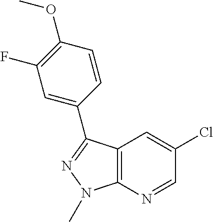 [SMILES: COc1ccc(-c2nn(C)c3ncc(Cl)cc23)cc1F] | | |
| A.7 | 5a | 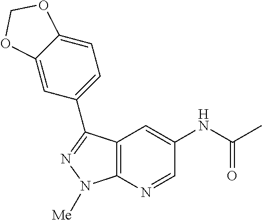 [SMILES: CC(=O)Nc1cnc2c(c1)c(-c1ccc3c(c1)OCO3)nn2C] | | |
| A.8 | 5a | 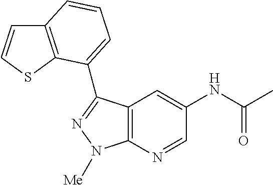 [SMILES: CC(=O)Nc1cnc2c(c1)c(-c1cccc3ccsc13)nn2C] | | |
| A.9 | 5a | 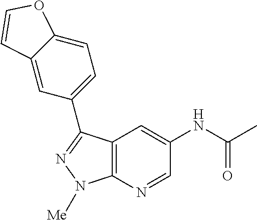 [SMILES: CC(=O)Nc1cnc2c(c1)c(-c1ccc3occc3c1)nn2C] | | |
| A.10 | 5a | 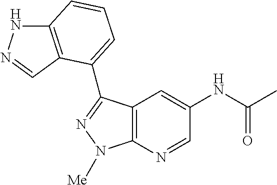 [SMILES: CC(=O)Nc1cnc2c(c1)c(-c1cccc3[nH]ncc13)nn2C] | | |
| A.11 | 5a | 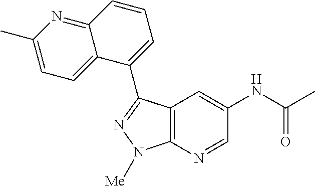 [SMILES: CC(=O)Nc1cnc2c(c1)c(-c1cccc3nc(C)ccc13)nn2C] | | |
| A.12 | 5a | 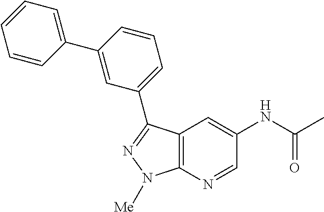 [SMILES: CC(=O)Nc1cnc2c(c1)c(-c1cccc(-c3ccccc3)c1)nn2C] | | |
| A.13 | 5a | 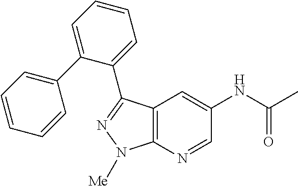 [SMILES: CC(=O)Nc1cnc2c(c1)c(-c1ccccc1-c1ccccc1)nn2C] | | |
| A.14 | 5a | 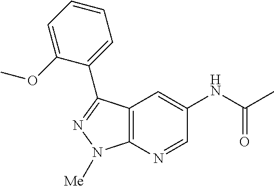 [SMILES: COc1ccccc1-c1nn(C)c2ncc(NC(C)=O)cc12] | | |
| A.15 | 5a | 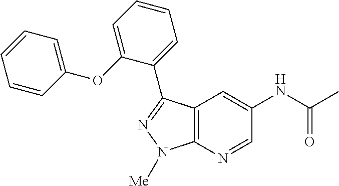 [SMILES: CC(=O)Nc1cnc2c(c1)c(-c1ccccc1Oc1ccccc1)nn2C] | | |
| A.16 | 5a | 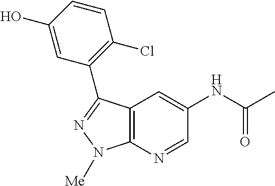 [SMILES: CC(=O)Nc1cnc2c(c1)c(-c1cc(O)ccc1Cl)nn2C] | | |
| A.17 | 5a | 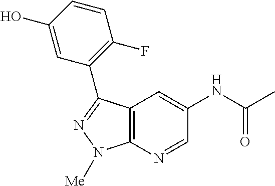 [SMILES: CC(=O)Nc1cnc2c(c1)c(-c1cc(O)ccc1F)nn2C] | | |
| A.18 | 5a | 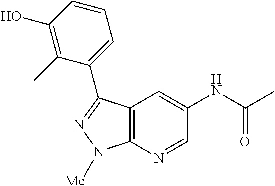 [SMILES: CC(=O)Nc1cnc2c(c1)c(-c1cccc(O)c1C)nn2C] | | |
| A.19 | 5a | 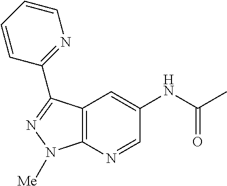 [SMILES: CC(=O)Nc1cnc2c(c1)c(-c1ccccn1)nn2C] | | |
| A.20 | 5a | 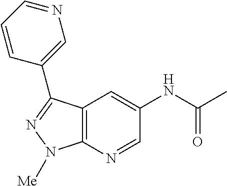 [SMILES: CC(=O)Nc1cnc2c(c1)c(-c1cccnc1)nn2C] | | |
| A.21 | 5a | 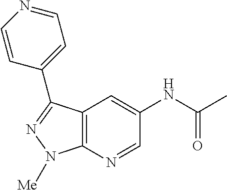 [SMILES: CC(=O)Nc1cnc2c(c1)c(-c1ccncc1)nn2C] | | |
| A.22 | 5a | 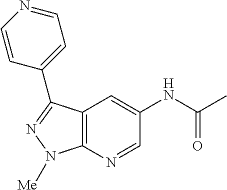 [SMILES: CC(=O)Nc1cnc2c(c1)c(-c1ccncc1)nn2C] | | |
| A.23 | 5a | 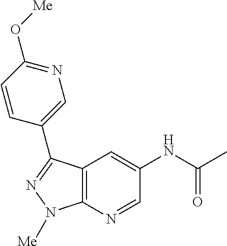 [SMILES: COc1ccc(-c2nn(C)c3ncc(NC(C)=O)cc23)cn1] | | |

### Examples B.1-B.12

Examples B.1-B.12 are prepared according to synthesis B.

| | | | Mass | HPLC Rt |
|---|---|---|---|---|
| # | Educt | Structure | [M + 1]⁺ | [min] |
| B.1 | 7 | 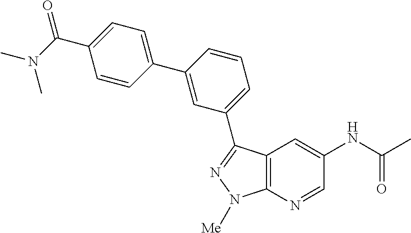 [SMILES: CC(=O)Nc1cnc2c(c1)c(-c1cccc(-c3ccc(C(=O)N(C)C)cc3)c1)nn2C] | | |
| B.2 | A.16 | 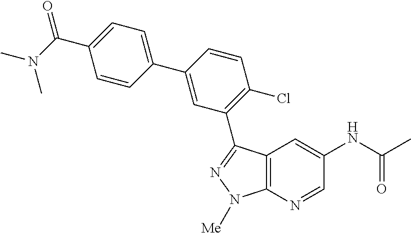 [SMILES: CC(=O)Nc1cnc2c(c1)c(-c1cc(-c3ccc(C(=O)N(C)C)cc3)ccc1Cl)nn2C] | | |
| B.3 | A.17 | 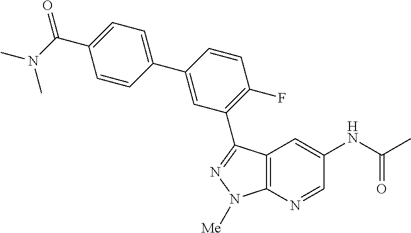 [SMILES: CC(=O)Nc1cnc2c(c1)c(-c1cc(-c3ccc(C(=O)N(C)C)cc3)ccc1F)nn2C] | | |
| B.4 | A.18 | 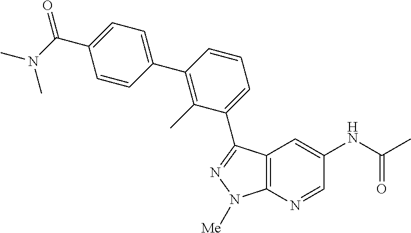 [SMILES: CC(=O)Nc1cnc2c(c1)c(-c1cccc(-c3ccc(C(=O)N(C)C)cc3)c1C)nn2C] | | |
| B.5 | 7 | 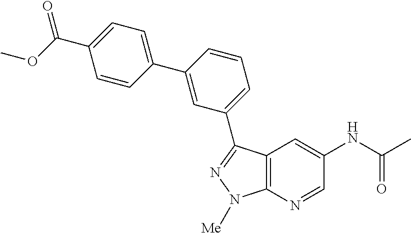 [SMILES: COC(=O)c1ccc(-c2cccc(-c3nn(C)c4ncc(NC(C)=O)cc34)c2)cc1] | | |
| B.6 | 7 | 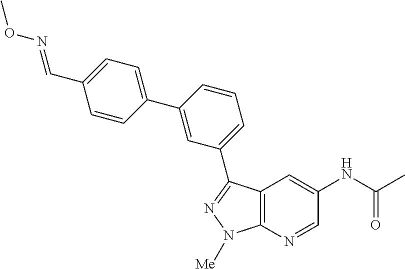 [SMILES: CO/N=C/c1ccc(-c2cccc(-c3nn(C)c4ncc(NC(C)=O)cc34)c2)cc1] | | |
| B.7 | 7 | 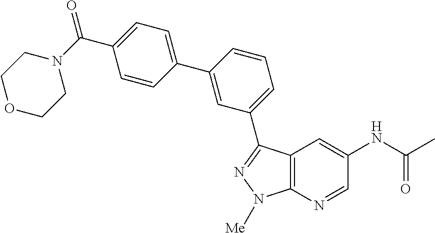 [SMILES: CC(=O)Nc1cnc2c(c1)c(-c1cccc(-c3ccc(C(=O)N4CCOCC4)cc3)c1)nn2C] | | |
| B.8 | 7 | 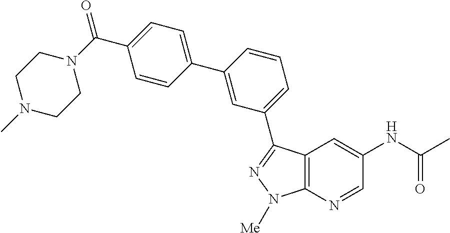 [SMILES: CC(=O)Nc1cnc2c(c1)c(-c1cccc(-c3ccc(C(=O)N4CCN(C)CC4)cc3)c1)nn2C] | | |
| B.9 | 7 | 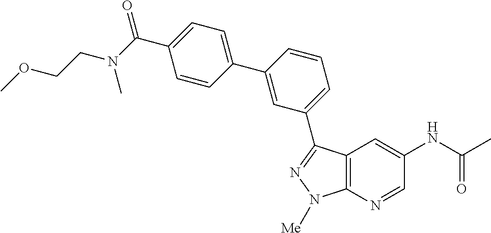 [SMILES: COCCN(C)C(=O)c1ccc(-c2cccc(-c3nn(C)c4ncc(NC(C)=O)cc34)c2)cc1] | | |
| B.10 | 7 | 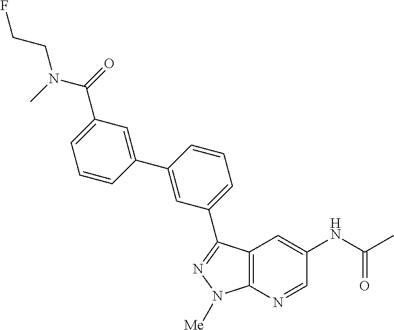 [SMILES: CC(=O)Nc1cnc2c(c1)c(-c1cccc(-c3cccc(C(=O)N(C)CCF)c3)c1)nn2C] | | |
| B.11 | 7 | 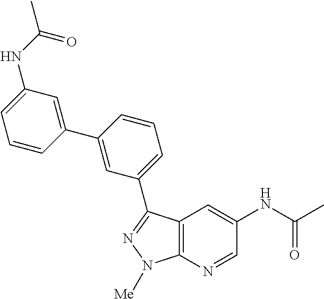 [SMILES: CC(=O)Nc1cccc(-c2cccc(-c3nn(C)c4ncc(NC(C)=O)cc34)c2)c1] | | |
| B.12 | 7 | 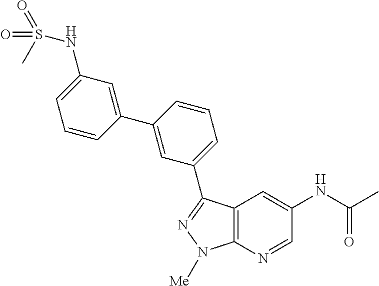 [SMILES: CC(=O)Nc1cnc2c(c1)c(-c1cccc(-c3cccc(NS(C)(=O)=O)c3)c1)nn2C] | | |

The Example that follows describes the biological activity of the compounds according to the invention without restricting the invention to this Example.

Inhibition of kinase activity by compounds is monitored by measurement of the phosphorylation of the substrate phosphatidylinositol-4,5-biphosphate, contained in a lipid blend, by recombinant PI3 kinase. In 96 well microtiter plates, compounds are serially diluted in assay buffer and mixed with lipid vesicles, PI3 kinase and phosphotyrosine-PDGFR-peptide used as kinase activator. The mixture is incubated for 20 min. at RT. Subsequently, the kinase reaction is started with ATP and 33P-ATP as a tracer. After a 2 h incubation at RT, the reaction is filtered to remove unbound radioactivity, and labelled phosphatidylinositol-3,4,5-triphosphate is measured in a Wallac Trilux Microbeta Counter. As positive control serve wells containing vehicle control showing non-inhibited kinase activity. Determination of IC50 values are carried out using GraphPad Prism 3.0. The IC50 values are below 10 μM for the compounds.

The substances of the present invention are PI3 kinase inhibitors. On account of their biological properties, the novel compounds of the general formula (I) and their isomers and their physiologically tolerated salts are suitable for treating diseases which are characterized by excessive or anomalous cell proliferation.

Such diseases include for example: viral infections (e.g. HIV and Kaposi's sarcoma); inflammatory and autoimmune diseases (e.g. colitis, arthritis, Alzheimer's disease, glomerulonephritis and wound healing); bacterial, fungal and/or parasitic infections; leukaemias, lymphomas and solid tumours (e.g. carcinomas and sarcomas), skin diseases (e.g. psoriasis); diseases based on hyperplasia which are characterised by an increase in the number of cells (e.g. fibroblasts, hepatocytes, bones and bone marrow cells, cartilage or smooth muscle cells or epithelial cells (e.g. endometrial hyperplasia)); bone diseases and cardiovascular diseases (e.g. restenosis and hypertrophy). They are also useful for protecting proliferating cells (e.g. hair, intestinal, blood and progenitor cells) from DNA damage caused by radiation, UV treatment and/or cytostatic treatment.

For example, the following cancers may be treated with compounds according to the invention, without being restricted thereto: brain tumours such as for example acoustic neurinoma, astrocytomas such as pilocytic astrocytomas, fibrillary astrocytoma, protoplasmic astrocytoma, gemistocytary astrocytoma, anaplastic astrocytoma and glioblastoma, brain lymphomas, brain metastases, hypophyseal tumour such as prolactinoma, HGH (human growth hormone) producing tumour and ACTH producing tumour (adrenocorticotropic hormone), craniopharyngiomas, medulloblastomas, meningeomas and oligodendrogliomas; nerve tumours (neoplasms) such as for example tumours of the vegetative nervous system such as neuroblastoma sympathicum, ganglioneuroma, paraganglioma (pheochromocytoma, chromaffinoma) and glomus-caroticum tumour, tumours on the peripheral nervous system such as amputation neuroma, neurofibroma, neurinoma (neurilemmoma, Schwannoma) and malignant Schwannoma, as well as tumours of the central nervous system such as brain and bone marrow tumours; intestinal cancer such as for example carcinoma of the rectum, colon, anus, small intestine and duodenum; eyelid tumours such as basalioma or basal cell carcinoma; pancreatic cancer or carcinoma of the pancreas; bladder cancer or carcinoma of the bladder; lung cancer (bronchial carcinoma) such as for example small-cell bronchial carcinomas (oat cell carcinomas) and non-small cell bronchial carcinomas such as plate epithelial carcinomas, adenocarcinomas and large-cell bronchial carcinomas; breast cancer such as for example mammary carcinoma such as infiltrating ductal carcinoma, colloid carcinoma, lobular invasive carcinoma, tubular carcinoma, adenocystic carcinoma and papillary carcinoma; non-Hodgkin's lymphomas (NHL) such as for example Burkitt's lymphoma, low-malignancy non-Hodgkin's lymphomas (NHL) and mucosis fungoides; uterine cancer or endometrial carcinoma or corpus carcinoma; CUP syndrome (Cancer of Unknown Primary); ovarian cancer or ovarian carcinoma such as mucinous, endometrial or serous cancer; gall bladder cancer; bile duct cancer such as for example Klatskin tumour; testicular cancer such as for example seminomas and non-seminomas; lymphoma (lymphosarcoma) such as for example malignant lymphoma, Hodgkin's disease, non-Hodgkin's lymphomas (NHL) such as chronic lymphatic leukaemia, leukaemic reticuloendotheliosis, immunocytoma, plasmocytoma (multiple myeloma), immunoblastoma, Burkitt's lymphoma, T-zone mycosis fungoides, large-cell anaplastic lymphoblastoma and lymphoblastoma; laryngeal cancer such as for example tumours of the vocal cords, supra-glottal, glottal and subglottal laryngeal tumours; bone cancer such as for example osteochondroma, chondroma, chondroblastoma, chondromyxoid fibroma, osteoma, osteoid osteoma, osteoblastoma, eosinophilic granuloma, giant cell tumour, chondrosarcoma, osteosarcoma, Ewing's sarcoma, reticulo-sarcoma, plasmocytoma, giant cell tumour, fibrous dysplasia, juvenile bone cysts and aneurysmatic bone cysts; head and neck tumours such as for example tumours of the lips, tongue, floor of the mouth, oral cavity, gums, palate, salivary glands, throat, nasal cavity, paranasal sinuses, larynx and middle ear; liver cancer such as for example liver cell carcinoma or hepatocellular carcinoma (HCC); leukaemias, such as for example acute leukaemias such as acute lymphatic/lymphoblastic leukaemia (ALL), acute myeloid leukaemia (AML); chronic leukaemias such as chronic lymphatic leukaemia (CLL), chronic myeloid leukaemia (CML); stomach cancer or gastric carcinoma such as for example papillary, tubular and mucinous adenocarcinoma, signet ring cell carcinoma, adenosquamous carcinoma, small-cell carcinoma and undifferentiated carcinoma; melanomas such as for example superficially spreading, nodular, lentigo-maligna and acral-lentiginous melanoma; renal cancer such as for example kidney cell carcinoma or hypernephroma or Grawitz's tumour; oesophageal cancer or carcinoma of the oesophagus; penile cancer; prostate cancer; throat cancer or carcinomas of the pharynx such as for example nasopharynx carcinomas, oropharynx carcinomas and hypopharynx carcinomas; retinoblastoma; vaginal cancer or vaginal carcinoma; plate epithelial carcinomas, adenocarcinomas, in situ carcinomas, malignant melanomas and sarcomas; thyroid carcinomas such as for example papillary, follicular and medullary thyroid carcinoma, as well as anaplastic carcinomas; spinalioma, epidormoid carcinoma and plate epithelial carcinoma of the skin; thymomas, cancer of the urethra and cancer of the vulva.

The new compounds may be used for the prevention, short-term or long-term treatment of the above-mentioned diseases, optionally also in combination with radiotherapy or other “state-of-the-art” compounds, such as e.g. cytostatic or cytotoxic substances, cell proliferation inhibitors, anti-angiogenic substances, steroids or antibodies.

The compounds of general formula (I) may be used on their own or in combination with other active substances according to the invention, optionally also in combination with other pharmacologically active substances.

Chemotherapeutic agents which may be administered in combination with the compounds according to the invention include, without being restricted thereto, hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxyprogesterone, octreotide), aromatase inhibitors (e.g. anastrozole, letrozole, liarozole, vorozole, exemestane, atamestane), LHRH agonists and antagonists (e.g. goserelin acetate, luprolide), inhibitors of growth factors (growth factors such as for example “platelet derived growth factor” and “hepatocyte growth factor”, inhibitors are for example “growth factor” antibodies, “growth factor receptor” antibodies and tyrosinekinase inhibitors, such as for example gefitinib, imatinib, lapatinib and trastuzumab); antimetabolites (e.g. antifolates such as methotrexate, raltitrexed, pyrimidine analogues such as 5-fluorouracil, capecitabin and gemcitabin, purine and adenosine analogues such as mercaptopurine, thioguanine, cladribine and pentostatin, cytarabine, fludarabine); antitumour antibiotics (e.g. anthracyclins such as doxorubicin, daunorubicin, epirubicin and idarubicin, mitomycin-C, bleomycin, dactinomycin, plicamycin, streptozocin); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); topoisomerase inhibitors (e.g. epipodophyllotoxins such as for example etoposide and etopophos, teniposide, amsacrin, topotecan, irinotecan, mitoxantron) and various chemotherapeutic agents such as amifostin, anagrelid, clodronat, filgrastin, interferon alpha, leucovorin, rituximab, procarbazine, levamisole, mesna, mitotane, pamidronate and porfimer.

Suitable preparations include for example tablets, capsules, suppositories, solutions,—particularly solutions for injection (s.c., i.v., i.m.) and infusion—elixirs, emulsions or dispersible powders. The content of the pharmaceutically active compound(s) should be in the range from 0.1 to 90 wt.-%, preferably 0.5 to 50 wt.-% of the composition as a whole, i.e. in amounts which are sufficient to achieve the dosage range specified below. The doses specified may, if necessary, be given several times a day.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose) emulsifiers (e.g. lignin, spent sulphite liquors, methylcellulose, starch and polyvinylpyrrolidone) and lubricants (e.g. magnesium stearate, talc, stearic acid and sodium lauryl sulphate).

The preparations are administered by the usual methods, preferably by oral or transdermal route, most preferably by oral route. For oral administration the tablets may, of course contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

For parenteral use, solutions of the active substances with suitable liquid carriers may be used.

The dosage for intravenous use is from 1-1000 mg per hour, preferably between 5 and 500 mg per hour.

However, it may sometimes be necessary to depart from the amounts specified, depending on the body weight, the route of administration, the individual response to the drug, the nature of its formulation and the time or interval over which the drug is administered. Thus, in some cases it may be sufficient to use less than the minimum dose given above, whereas in other cases the upper limit may have to be exceeded. When administering large amounts it may be advisable to divide them up into a number of smaller doses spread over the day.

The formulation examples that follow illustrate the present invention without restricting its scope:

### Examples of Pharmaceutical Formulations

| | | |
|---|---|---|
| A) | Tablets | per tablet |

| | |
|---|---|
| active substance according to formula (1) | 100 mg |
| lactose | 140 mg |
| corn starch | 240 mg |
| polyvinylpyrrolidone | 15 mg |
| magnesium stearate | 5 mg |
| | 500 mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

| | | |
|---|---|---|
| B) | Tablets | per tablet |

| | |
|---|---|
| active substance according to formula (1) | 80 mg |
| lactose | 55 mg |
| corn starch | 190 mg |
| microcrystalline cellulose | 35 mg |
| polyvinylpyrrolidone | 15 mg |
| sodium-carboxymethyl starch | 23 mg |
| magnesium stearate | 2 mg |
| | 400 mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodiumcarboxymethyl starch and the magnesium stearate are added and mixed in and the mixture is compressed to form tablets of a suitable size.

| | |
|---|---|
| C) | Ampoule solution |

| | | |
|---|---|---|
| active substance according to formula (1) | 50 | mg |
| sodium chloride | 50 | mg |
| water for inj. | 5 | mL |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 5 mg, 25 mg and 50 mg of active substance.

1. A compound of formula (I) 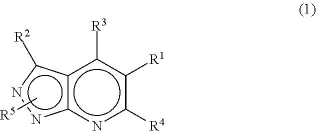
wherein
R¹ is selected from the group consisting of C₁₋₆alkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl, all the above-mentioned groups optionally being substituted by one or more identical or different R⁶; or
R¹ is selected from the group consisting of, —OR^{c}, C₁₋₃haloalkyloxy, —OCF₃, —SR^{c}, —NR^{c}R^{c}, —ONR^{c}R^{c}, —N(OR^{c})R^{c}, —N(R^{g})NR^{c}R^{c}, halogen, —CF₃, —CN, —NC, —OCN, —SCN, —NO, —NO₂, ═N₂, —N₃, —S(O)R^{c}, —S(O)OR^{c}, —S(O)₂R^{c}, —S(O)₂OR^{c}, —S(O)NR^{c}R^{c}, —S(O)₂NR^{c}R^{c}, —OS(O)R^{c}, —OS(O)₂R^{c}, —OS(O)₂OR^{c}, —OS(O)NR^{c}R^{c}, —OS(O)₂NR^{c}R^{c}, —C(O)R^{c}, —C(O)OR^{c}, —C(O)SR^{c}, —C(O)NR^{c}R^{c}, —C(O)N(R^{g})NR^{c}R^{c}, —C(O)N(R^{g})OR^{c}, —C(NR^{g})NR^{c}R^{c}, —C(NOH)R^{c}, —C(NOH)NR^{c}R^{c}, —OC(O)R^{c}, —OC(O)OR^{c}, —OC(O)SR^{c}, —OC(O)NR^{c}R^{c}, —OC(NR^{g})NR^{c}R^{c}, —SC(O)R^{c}, —SC(O)OR^{c}, —SC(O)NR^{c}R^{c}, —SC(NR^{g})NR^{c}R^{c}, —N(R^{g})C(O)R^{c}, —N[C(O)R^{c}]₂, —N(OR^{g})C(O)R^{c}, —N(R^{g})C(NR^{g})R^{c}, —N(R^{g})N(R^{g})C(O)R^{c}, —N[C(O)R^{c}]NR^{c}R^{c}, —N(R^{g})C(S)R^{c}, —N(R^{g})S(O)R^{c}, —N(R^{g})S(O)OR^{c}, —N(R^{g})S(O)₂R^{c}, —N[S(O)₂R^{c}]₂, —N(R^{g})S(O)₂OR^{c}, —N(R^{g})S(O)₂NR^{c}R^{c}, —N(R^{g})[S(O)₂]₂R^{c}, —N(R^{g})C(O)OR^{c}, —N(R^{g})C(O)SR^{c}, —N(R^{g})C(O)NR^{c}R^{c}, —N(R^{g})C(O)NR^{g}NR^{c}R^{c}, —N(R^{g})N(R^{g})C(O)NR^{c}R^{c}, —N(R^{g})C(S)NR^{c}R^{c}, —[N(R^{g})C(O)]₂R^{c}, —N(R^{g})[C(O)]₂R^{c}, —N{[C(O)]₂R^{c}}₂, —N(R^{g})[C(O)]₂OR^{c}, —N(R^{g})[C(O)]₂NR^{c}R^{c}, —N{[C(O)]₂OR^{c}}₂, —N{[C(O)]₂NR^{c}R^{c}}₂, —[N(R^{g})C(O)]₂OR^{c}, —N(R^{g})C(NR^{c})OR^{c}, —N(R^{g})C(NOH)R^{c}, —N(R^{g})C(NR^{g})SR^{c} and —N(R^{g})C(NR^{g})NR^{c}R^{c}, and —N═C(R^{g})NR^{c}R^{c}; and
R² denotes a group, optionally substituted by one or more R⁶, selected from among C₃₋₁₀cycloalkyl, 3-8-membered heterocycloalkyl, C₆₋₁₅aryl and 5-12-membered Heteroaryl; and wherein R² is not benzimidazolyl;
R³ and R⁴ independently from each other denotes hydrogen, R^{a} or R^{b},
R⁵ is selected from the group consisting of C₁₋₆alkyl, C₃₋₁₀cycloalkyl, C₄₋₁₆cycloalkylalkyl, C₇₋₁₆arylalkyl, 6-18 membered heteroarylalkyl, 3-14 membered heterocycloalkyl and 4-14 membered heterocycloalkylalkyl, all the above-mentioned groups optionally being substituted by one or more identical or different R^{f}, and R⁵ can be placed on any of the 2 N of the pyrazole ring; and
each R⁶ denotes a group selected from among R^{a}, R^{b} and R^{a} substituted by one or more identical or different R^{c} and/or R^{b}
each R^{a} independently of one another denotes hydrogen or a group optionally substituted by one or more identical or different R^{b} and/or R^{c}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl,
each R^{b} denotes a suitable group and is selected independently of one another from among ═O, —OR^{c}, C₁₋₃haloalkyloxy, —OCF₃, ═S, —SR^{c}, ═NR^{c}, ═NOR^{c}, ═NNR^{c}R^{c}, ═NN(R^{g})C(O)NR^{c}R^{c}, —NR^{c}R^{c}, —ONR^{c}R^{c}, —N(OR^{c})R^{c}, —N(R^{g})NR^{c}R^{c}, halogen, —CF₃, —CN, —NC, —OCN, —SCN, —NO, —NO₂, ═N₂, —N₃, —S(O)R^{c}, —S(O)OR^{c}, —S(O)₂R^{c}, —S(O)₂OR^{c}, —S(O)NR^{c}R^{c}, —S(O)₂NR^{c}R^{c}, —OS(O)R^{c}, —OS(O)₂R^{c}, —OS(O)₂OR^{c}, —OS(O)NR^{c}R^{c}, —OS(O)₂NR^{c}R^{c}, —C(O)R^{c}, —C(O)OR^{c}, —C(O)SR^{c}, —C(O)NR^{c}R^{c}, —C(O)N(R^{g})NR^{c}R^{c}, —C(O)N(R^{g})OR^{c}, —C(NR^{g})NR^{c}R^{c}, —C(NOH)R^{c}, —C(NOH)NR^{c}R^{c}, —OC(O)R^{c}, —OC(O)OR^{c}, —OC(O)SR^{c}, —OC(O)NR^{c}R^{c}, —OC(NR^{g})NR^{c}R^{c}, —SC(O)R^{c}, —SC(O)OR^{c}, —SC(O)NR^{c}R^{c}, —SC(NR^{g})NR^{c}R^{c}, —N(R^{g})C(O)R^{c}, —N[C(O)R^{c}]₂, —N(OR)C(O)R^{c}, —N(R^{g})C(NR^{g})R^{c}, —N(R^{g})N(R^{g})C(O)R^{c}, —N[C(O)R^{c}]NR^{c}R^{c}, —N(R^{g})C(S)R^{c}, —N(R^{g})S(O)R^{c}, —N(R^{g})S(O)OR^{c}, —N(R^{g})S(O)₂R^{c}, —N[S(O)₂R^{c}]₂, —N(R^{g})S(O)₂OR^{c}, —N(R^{g})S(O)₂NR^{c}R^{c}, —N(R^{g})[S(O)₂]₂R^{c}, —N(R^{g})C(O)OR^{c}, —N(R^{g})C(O)SR^{c}, —N(R^{g})C(O)NR^{c}R^{c}, —N(R^{g})C(O)NR^{g}NR^{c}R^{c}, —N(R^{g})N(R^{g})C(O)NR^{c}R^{c}, —N(R^{g})C(S)NR^{c}R^{c}, —[N(R^{g})C(O)]₂R^{c}, —N(R^{g})[C(O)]₂R^{c}, —N{[C(O)]₂R^{c}}₂, —N(R^{g}) [C(O)]₂OR^{c}, —N(R^{g}) [C(O)]₂NR^{c}R^{c}, —N{[C(O)]₂OR^{c}}₂, —N{[C(O)]₂NR^{c}R^{c}}₂, —[N(R^{g})C(O)]₂OR^{c}, —N(R^{g})C(NR^{g})OR^{c}, —N(R^{g})C(NOH)R^{c}, —N(R^{g})C(NR^{g})SR^{c}, —N(R^{g})C(NR^{g})NR^{c}R^{c} and —N═C(R^{g})NR^{c}R^{c} and
each R^{c} independently of one another denotes hydrogen or a group optionally substituted by one or more identical or different R^{d} and/or R^{e}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl, and
each R^{d} denotes a suitable group and is selected independently of one another from among ═O, —OR^{e}, C₁₋₃haloalkyloxy, —OCF₃, ═S, —SR^{e}, ═NR^{e}, ═NOR^{e}, ═NNR^{e}R^{e}, ═NN(R^{g})C(O)NR^{e}R^{e}, —NR^{e}R^{e}, —ONR^{e}R^{e}, —N(R^{g})NR^{e}R^{e}, halogen, —CF₃, —CN, —NC, —OCN, —SCN, —NO, —NO₂, ═N₂, —N₃, —S(O)R^{e}, —S(O)OR^{e}, —S(O)₂R^{e}, —S(O)₂OR^{e}, —S(O)NR^{e}R^{e}, —S(O)₂NR^{e}R^{e}, —OS(O)R^{e}, —OS(O)₂R^{e}, —OS(O)₂OR^{e}, —OS(O)NR^{e}R^{e}, —OS(O)₂NR^{e}R^{e}, —C(O)R^{e}, —C(O)OR^{e}, —C(O)SR^{e}, —C(O)NR^{e}R^{e}, —C(O)N(R^{g})NR^{e}R^{e}, —C(O)N(R)OR^{e}, —C(NR^{g})NR^{e}R^{e}, —C(NOH)R^{e}, —C(NOH)NR^{e}R^{e}, —OC(O)R^{e}, —OC(O)OR^{e}, —OC(O)SR^{e}, —OC(O)NR^{e}R^{e}, —OC(NR^{g})NR^{e}R^{e}, —SC(O)R^{e}, —SC(O)OR^{e}, —SC(O)NR^{e}R^{e}, —SC(NR^{g})NR^{e}R^{e}, —N(R^{g})C(O)R^{e}, —N[C(O)R^{e}]₂, —N(OR^{g})C(O)R^{e}, —N(R)C(NR^{g})R^{e}, —N(R^{g})N(R^{g})C(O)R^{e}, —N[C(O)R^{e}]NR^{e}R^{e}, —N(R^{g})C(S)R^{e}, —N(R)S(O)R^{e}, —N(R^{g})S(O)OR^{e}—N(R^{g})S(O)₂R^{e}, —N[S(O)₂R^{e}]₂, —N(R^{g})S(O)₂OR^{e}, —N(R^{g})S(O)₂NR^{e}R^{e}, —N(R^{g})[S(O)₂]₂R^{e}, —N(R^{g})C(O)OR^{e}, —N(R^{g})C(O)SR^{e}, —N(R^{g})C(O)NR^{e}R^{e}, —N(R^{g})C(O)NR^{g}NR^{e}R^{e}, —N(R^{g})N(R^{g})C(O)NR^{e}R^{e}, —N(R^{g})C(S)NR^{e}R^{e}, —[N(R^{g})C(O)]₂R^{e}, —N(R^{g})[C(O)]₂R^{e}, —N{[C(O)]₂R^{e}}₂, —N(R^{g}) [C(O)]₂OR^{e}, —N(R^{g}) [C(O)]₂NR^{e}R^{e}, —N{[C(O)]₂OR^{e}}₂, —N{[C(O)]₂NR^{e}R^{e}}₂, —[N(R^{g})C(O)]₂OR^{e}, —N(R^{g})C(NR^{g})OR^{e}, —N(R^{g})C(NOH)R^{e}, —N(R^{g})C(NR^{g})SR^{e}, —N(R^{g})C(NR^{g})NR^{e}R^{e} and —N═C(R^{g})NR^{e}R^{e}
each R^{e} independently of one another denotes hydrogen or a group optionally substituted by one or more identical or different R^{f} and/or R^{g}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl, and
each R^{f} denotes a suitable group and in each case is selected independently of one another from among ═O, —OR^{g}, C₁₋₃haloalkyloxy, —OCF₃, ═S, —SR^{g}, ═NR^{g}, ═NOR^{g}, ═NNR^{g}R^{g}, ═NN(R^{h})C(O)NR^{g}R^{g}, —NR^{g}R^{g}, —ONR^{g}R^{g}, —N(R^{h})NR^{g}R^{g}, halogen, —CF₃, —CN, —NC, —OCN, —SCN, —NO, —NO₂, ═N₂, —N₃, —S(O)R^{g}, —S(O)OR^{g}, —S(O)₂R^{g}, —S(O)₂OR^{g}, —S(O)NR^{g}R^{g}, —S(O)₂NR^{g}R^{g}, —OS(O)R^{g}, —OS(O)₂R^{g}, —OS(O)₂R^{g}, —OS(O)NR^{g}R^{g}, —OS(O)₂NR^{g}R^{g}, —C(O)R^{g}, —C(O)OR^{g}, —C(O)SR^{g}, —C(O)NR^{g}R^{g}, —C(O)N(R^{h})NR^{g}R^{g}, —C(O)N(R^{h})OR^{g}, —C(NR^{h})NR^{g}R^{g}, —C(NOH)R^{g}, —C(NOH)NR^{g}R^{g}, —OC(O)R^{g}, —OC(O)OR^{g}, —OC(O)SR^{g}, —OC(O)NR^{g}R^{g}, —OC(NR^{h})NR^{g}R^{g}, —SC(O)R^{g}, —SC(O)OR^{g}, —SC(O)NR^{g}R^{g}, —SC(NR^{h})NR^{g}R^{g}, —N(R^{h})C(O)R^{g}, —N[C(O)R^{g}]₂, —N(OR^{h})C(O)R^{g}, —N(R^{h})C(NR^{h})R^{g}, —N(R^{h})N(R^{h})C(O)R^{g}, —N[C(O)R]NR^{g}R^{g}, —N(R^{h})C(S)R^{g}, —N(R^{h})S(O)R^{g}, —N(R^{h})S(O)OR^{g}, —N(R^{h})S(O)₂R^{g}, —N[S(O)₂R^{g}]₂, —N(R^{h})S(O)₂R^{g}, —N(R^{h})S(O)₂NR^{g}R^{g}, —N(R^{h})[S(O)₂]₂R^{g}, —N(R^{h})C(O)OR^{g}, —N(R^{h})C(O)SR^{g}, —N(R^{h})C(O)NR^{g}R^{g}, —N(R^{h})C(O)NR^{h}NR^{g}R^{g}, —N(R^{h})N(R^{h})C(O)NR^{g}R^{g}, —N(R^{h})C(S)NR^{g}R^{g}, —[N(R^{h})C(O)]₂R^{g}, —N(R^{h})[C(O)]₂R^{g}, —N{[C(O)]₂R^{g}}₂, —N(R^{h})[C(O)]₂OR^{g}, —N(R^{h})[C(O)]₂NR^{g}R^{g}, —N{[C(O)]₂OR^{g}}₂, —N{[C(O)]₂NR^{g}R^{g}}₂, —[N(R^{h})C(O)]₂OR^{g}, —N(R^{h})C(NR^{h})OR^{g}, —N(R^{h})C(NOH)R^{g}, —N(R^{h})C(NR^{h})SR^{g}, —N(R^{h})C(NR^{h})NR^{g}R^{g}; and —N═C(R^{h})NR^{h}R^{h}; and
each R^{g} independently of one another denotes hydrogen or a group optionally substituted by one or more identical or different R^{h}, selected from among C₁₋₆alkyl, 2-6 membered heteroalkyl, C₁₋₆haloalkyl, C₃₋₁₀ cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl; and
each R^{h} is selected independently of one another from among hydrogen, C₁₋₆alkyl, 2-6 membered heteroalkyl, C₁₋₆haloalkyl, C₃₋₁₀ cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl, a tautomer thereof, a racemate thereof, an enantiomer thereof, a diastereomer thereof, or a mixture of any of the foregoing, or a pharmacologically acceptable salt thereof.

2. The compound according to claim 1, wherein R³ denotes hydrogen.

3. The compound according to claim 1, wherein R⁴ denotes hydrogen.

4. The compound according to claim 1, wherein R⁵ denotes C₁₋₃alkyl.

5. A pharmaceutical preparation comprising as active substance one or more compounds of formula (I) according to claim 1 and one or more conventional excipients or carriers.

6. The pharmaceutical preparation according to claim 5 comprising at least one other cytostatic or cytotoxic active substance different from any compounds according to claim 1.

7. A method for the prevention or treatment of cancer, infections, inflammatory diseases or autoimmune diseases which comprises administering a therapeutically effective amount of a compound according to claim 1.

8. A compound of formula (I) 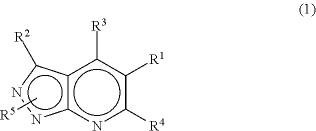
wherein
R¹ is selected from the group consisting of C₁₋₆alkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl, all the above-mentioned groups optionally being substituted by one or more identical or different R⁶; or
R¹ is selected from the group consisting of, —OR^{c}, C₁₋₃haloalkyloxy, —OCF₃, —SR^{c}, —NR^{c}R^{c}, —ONR^{c}R^{c}, —N(OR^{c})R^{c}, —N(R^{g})NR^{c}R^{c}, halogen, —CF₃, —CN, —NC, —OCN, —SCN, —NO, —NO₂, ═N₂, —N₃, —S(O)R^{c}, —S(O)OR^{c}, —S(O)₂R^{c}, —S(O)₂OR^{c}, —S(O)NR^{c}R^{c}, —S(O)₂NR^{c}R^{c}, —OS(O)R^{c}, —OS(O)₂R^{c}, —OS(O)₂OR^{c}, —OS(O)NR^{c}R^{c}, —OS(O)₂NR^{c}R^{c}, —C(O)R^{c}, —C(O)OR^{c}, —C(O)SR^{c}, —C(O)NR^{c}R^{c}, —C(O)N(R^{g})NR^{c}R^{c}, —C(O)N(R^{g})OR^{c}, —C(NR^{g})NR^{c}R^{c}, —C(NOH)R^{c}, —C(NOH)NR^{c}R^{c}, —OC(O)R^{c}, —OC(O)OR^{c}, —OC(O)SR^{c}, —OC(O)NR^{c}R^{c}, —OC(NR^{g})NR^{c}R^{c}, —SC(O)R^{c}, —SC(O)OR^{c}, —SC(O)NR^{c}R^{c}, —SC(NR^{g})NR^{c}R^{c}, —N(R^{g})C(O)R^{c}, —N[C(O)R^{c}]₂, —N(OR^{g})C(O)R^{c}, —N(R^{g})C(NR^{g})R^{c}, —N(R^{g})N(R)C(O)R^{c}, —N[C(O)R^{c}]NR^{c}R^{c}, —N(R^{g})C(S)R^{c}, —N(R^{g})S(O)R^{c}, —N(R^{g})S(O)OR^{c}, —N(R^{g})S(O)₂R^{c}, —N[S(O)₂R^{c}]₂, —N(R^{g})S(O)₂OR^{c}, —N(R^{g})S(O)₂NR^{c}R^{c}, —N(R⁾[S(O)₂]₂R^{c}, —N(R^{g})C(O)OR^{c}, —N(R^{g})C(O)SR^{c}, —N(R^{g})C(O)NR^{c}R^{c}, —N(R⁾C(O)NR^{g}NR^{c}R^{c}, —N(R^{g})N(R^{g})C(O)NR^{c}R^{c}, —N(R^{g})C(S)NR^{c}R^{c}, —[N(R^{g})C(O)]₂R^{c}, —N(R)[C(O)]₂R^{c}, —N{[C(O)]₂R^{c}}₂, —N(R^{g})[C(O)]₂OR^{c}, —N(R^{g})[C(O)]₂NR^{c}R^{c}, —N{[C(O)]₂OR^{c}}₂, —N{[C(O)]₂NR^{c}R^{c}}₂, —[N(R^{g})C(O)]₂OR^{c}, —N(R^{g})C(NR^{g})OR^{c}, —N(R^{g})C(NOH)R^{c}, —N(R^{g})C(NR^{g})SR^{c} and —N(R^{g})C(NR^{g})NR^{c}R^{c}, and —N═C(R^{g})NR^{c}R^{c}; and
R² denotes a group, optionally substituted by one or more R⁶, selected from among C₃₋₁₀cycloalkyl, 3-8-membered heterocycloalkyl, C₆₋₁₅aryl and 5-12-membered Heteroaryl; and wherein R² is not benzimidazolyl;
R³ and R⁴ independently from each other denotes hydrogen, R^{a} or R^{b},
R⁵ is selected from the group consisting of C₁₋₆alkyl, C₃₋₁₀cycloalkyl, C₄₋₁₆cycloalkylalkyl, C₇₋₁₆arylalkyl, 6-18 membered heteroarylalkyl, 3-14 membered heterocycloalkyl and 4-14 membered heterocycloalkylalkyl, all the above-mentioned groups optionally being substituted by one or more identical or different R^{f}, and R⁵ can be placed on any of the 2 N of the pyrazole ring; and
each R⁶ denotes a group selected from among R^{a}, R^{b} and R^{a} substituted by one or more identical or different R^{c} and/or R^{b};
each R^{a} independently of one another denotes hydrogen or a group optionally substituted by one or more identical or different R^{b} and/or R^{c}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl,
each R^{b} denotes a suitable group and is selected independently of one another from among ═O, —OR^{c}, C₁₋₃haloalkyloxy, —OCF₃, ═S, —SR^{c}, ═NR^{c}, ═NOR^{c}, ═NNR^{c}R^{c}, ═NN(R^{g})C(O)NR^{c}R^{c}, —NR^{c}R^{c}, —ONR^{c}R^{c}, —N(OR^{c})R^{c}, —N(R^{g})NR^{c}R^{c}, halogen, —CF₃, —CN, —NC, —OCN, —SCN, —NO, —NO₂, ═N₂, —N₃, —S(O)R^{c}, —S(O)OR^{c}, —S(O)₂R^{c}, —S(O)₂OR^{c}, —S(O)NR^{c}R^{c}, —S(O)₂NR^{c}R^{c}, —OS(O)R^{c}, —OS(O)₂R^{c}, —OS(O)₂OR^{c}, —OS(O)NR^{c}R^{c}, —OS(O)₂NR^{c}R^{c}, —C(O)R^{c}, —C(O)OR^{c}, —C(O)SR^{c}, —C(O)NR^{c}R^{c}, —C(O)N(R^{g})NR^{c}R^{c}, —C(O)N(R^{g})OR^{c}, —C(NR^{g})NR^{c}R^{c}, —C(NOH)R^{c}, —C(NOH)NR^{c}R^{c}, —OC(O)R^{c}, —OC(O)OR^{c}, —OC(O)SR^{c}, —OC(O)NR^{c}R^{c}, —OC(NR^{g})NR^{c}R^{c}, —SC(O)R^{c}, —SC(O)OR^{c}, —SC(O)NR^{c}R^{c}, —SC(NR^{g})NR^{c}R^{c}, —N(R^{g})C(O)R^{c}, —N[C(O)R^{c}]₂, —N(OR)C(O)R^{c}, —N(R^{g})C(NR^{g})R^{c}, —N(R^{g})N(R^{g})C(O)R^{c}, —N[C(O)R^{c}]NR^{c}R^{c}, —N(R^{g})C(S)R^{c}, —N(R^{g})S(O)R^{c}, —N(R^{g})S(O)OR^{c}, —N(R^{g})S(O)₂R^{c}, —N[S(O)₂R^{c}]₂, —N(R^{g})S(O)₂OR^{c}, —N(R^{g})S(O)₂NR^{c}R^{c}, —N(R^{g})[S(O)₂]₂R^{c}, —N(R^{g})C(O)OR^{c}, —N(R^{g})C(O)SR^{c}, —N(R^{g})C(O)NR^{c}R^{c}, —N(R^{g})C(O)NR^{g}NR^{c}R^{c}, —N(R^{g})N(R^{g})C(O)NR^{c}R^{c}, —N(R^{g})C(S)NR^{c}R^{c}, —[N(R^{g})C(O)]₂R^{c}, —N(R^{g})[C(O)]₂R^{c}, —N{[C(O)]₂R^{c}}₂, —N(R^{g}) [C(O)]₂OR^{c}, —N(R^{g}) [C(O)]₂NR^{c}R^{c}, —N{[C(O)]₂OR^{c}}₂, —N{[C(O)]₂NR^{c}R^{c}}₂, —[N(R^{g})C(O)]₂OR^{c}, —N(R^{g})C(NR^{g})OR^{c}, —N(R^{g})C(NOH)R^{c}, —N(R^{g})C(NR^{g})SR^{c}, —N(R^{g})C(NR^{g})NR^{c}R^{c} and —N═C(R^{g})NR^{c}R^{c} and
each R^{c} independently of one another denotes hydrogen or a group optionally substituted by one or more identical or different R^{d} and/or R^{e}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl, and
each R^{d} denotes a suitable group and is selected independently of one another from among ═O, —OR^{e}, C₁₋₃haloalkyloxy, —OCF₃, ═S, —SR^{e}, ═NR^{e}, ═NOR^{e}, ═NNR^{e}R^{e}, ═NN(R^{g})C(O)NR^{e}R^{e}, —NR^{e}R^{e}, —ONR^{e}R^{e}, —N(R^{g})NR^{e}R^{e}, halogen, —CF₃, —CN, —NC, —OCN, —SCN, —NO, —NO₂, ═N₂, —N₃, —S(O)R^{e}, —S(O)OR^{e}, —S(O)₂R^{e}, —S(O)₂OR^{e}, —S(O)NR^{e}R^{e}, —S(O)₂NR^{e}R^{e}, —OS(O)R^{e}, —OS(O)₂R^{e}, —OS(O)₂OR^{e}, —OS(O)NR^{e}R^{e}, —OS(O)₂NR^{e}R^{e}, —C(O)R^{e}, —C(O)OR^{e}, —C(O)SR^{e}, —C(O)NR^{e}R^{e}, —C(O)N(R^{g})NR^{e}R^{e}, —C(O)N(R^{g})OR^{e}, —C(NR^{g})NR^{e}R^{e}, —C(NOH)R^{e}, —C(NOH)NR^{e}R^{e}, —OC(O)R^{e}, —OC(O)OR^{e}, —OC(O)SR^{e}, —OC(O)NR^{e}R^{e}, —OC(NR^{g})NR^{e}R^{e}, —SC(O)R^{e}, —SC(O)OR^{e}, —SC(O)NR^{e}R^{e}, —SC(NR^{g})NR^{e}R^{e}, —N(R^{g})C(O)R^{e}, —N[C(O)R^{e}]₂, —N(OR^{g})C(O)R^{e}, —N(R)C(NR^{g})R^{e}, —N(R^{g})N(R^{g})C(O)R^{e}, —N[C(O)R^{e}]NR^{e}R^{e}, —N(R^{g})C(S)R^{e}, —N(R^{g})S(O)R^{e}, —N(R^{g})S(O)OR^{e}—N(R^{g})S(O)₂R^{e}, —N[S(O)₂R^{e}]₂, —N(R^{g})S(O)₂OR^{e}, —N(R^{g})S(O)₂NR^{e}R^{e}, —N(R^{g})[S(O)₂]₂R^{e}, —N(R^{g})C(O)OR^{e}, —N(R^{g})C(O)SR^{e}, —N(R^{g})C(O)NR^{e}R^{e}, —N(R^{g})C(O)NR^{g}NR^{e}R^{e}, —N(R^{g})N(R^{g})C(O)NR^{e}R^{e}, —N(R^{g})C(S)NR^{e}R^{e}, —[N(R^{g})C(O)]₂R^{e}, —N(R^{g})[C(O)]₂R^{e}, —N{[C(O)]₂R^{e}}₂, —N(R^{g}) [C(O)]₂OR^{e}, —N(R^{g}) [C(O)]₂NR^{e}R^{e}, —N{[C(O)]₂OR^{e}}₂, —N{[C(O)]₂NR^{e}R^{e}}₂, —[N(R^{g})C(O)]₂OR^{e}, —N(R^{g})C(NR^{g})OR^{e}, —N(R^{g})C(NOH)R^{e}, —N(R^{g})C(NR^{g})SR^{e}, —N(R^{g})C(NR^{g})NR^{e}R^{e} and —N═C(R^{g})NR^{e}R^{e}
each R^{e} independently of one another denotes hydrogen or a group optionally substituted by one or more identical or different R^{f} and/or R^{g}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl, and
each R^{f} denotes a suitable group and in each case is selected independently of one another from among ═O, —OR^{g}, C₁₋₃haloalkyloxy, —OCF₃, ═S, —SR^{g}, ═NR^{g}, ═NOR^{g}, ═NNR^{g}R^{g}, ═NN(R^{h})C(O)NR^{g}R^{g}, —NR^{g}R^{g}, —ONR^{g}R^{g}, —N(R^{h})NR^{g}R^{g}, halogen, —CF₃, —CN, —NC, —OCN, —SCN, —NO, —NO₂, ═N₂, —N₃, —S(O)R^{g}, —S(O)OR^{g}, —S(O)₂R^{g}, —S(O)₂OR^{g}, —S(O)NR^{g}R^{g}, —S(O)₂NR^{g}R^{g}, —OS(O)R^{g}, —OS(O)₂R^{g}, —OS(O)₂R^{g}, —OS(O)NR^{g}R^{g}, —OS(O)₂NR^{g}R^{g}, —C(O)R^{g}, —C(O)OR^{g}, —C(O)SR^{g}, —C(O)NR^{g}R^{g}, —C(O)N(R^{h})NR^{g}R^{g}, —C(O)N(R^{h})OR^{g}, —C(NR^{h})NR^{g}R^{g}, —C(NOH)R^{g}, —C(NOH)NR^{g}R^{g}, —OC(O)R^{g}, —OC(O)OR^{g}, —OC(O)SR^{g}, —OC(O)NR^{g}R^{g}, —OC(NR^{h})NR^{g}R^{g}, —SC(O)R^{g}, —SC(O)OR^{g}, —SC(O)NR^{g}R^{g}, —SC(NR^{h})NR^{g}R^{g}, —N(R^{h})C(O)R^{g}, —N[C(O)R^{g}]₂, —N(OR^{h})C(O)R^{g}, —N(R^{h})C(NR^{h})R^{g}, —N(R^{h})N(R^{h})C(O)R^{g}, —N[C(O)R^{g}]NR^{g}R^{g}, —N(R^{h})C(S)R^{g}, —N(R^{h})S(O)R^{g}, —N(R^{h})S(O)OR^{g}, —N(R^{h})S(O)₂R^{g}, —N[S(O)₂R^{g}]₂, —N(R^{h})S(O)₂R^{g}, —N(R^{h})S(O)₂NR^{g}R^{g}, —N(R^{h})[S(O)₂]₂R^{g}, —N(R^{h})C(O)OR^{g}, —N(R^{h})C(O)SR^{g}, —N(R^{h})C(O)NR^{g}R^{g}, —N(R^{h})C(O)NR^{h}NR^{g}R^{g}, —N(R^{h})N(R^{h})C(O)NR^{g}R^{g}, —N(R^{h})C(S)NR^{g}R^{g}, —[N(R^{h})C(O)]₂R^{g}, —N(R^{h})[C(O)]₂R^{g}, —N{[C(O)]₂R^{g}}₂, —N(R^{h})[C(O)]₂OR^{g}, —N(R^{h})[C(O)]₂NR^{g}R^{g}, —N{[C(O)]₂OR^{g}}₂, —N{[C(O)]₂NR^{g}R^{g}}₂, —[N(R^{h})C(O)]₂OR^{g}, —N(R^{h})C(NR^{h})OR^{g}, —N(R^{h})C(NOH)R^{g}, —N(R^{h})C(NR^{h})SR^{g}, —N(R^{h})C(NR^{h})NR^{g}R^{g}; and —N═C(R^{h})NR^{h}R^{h}; and
each R^{g} independently of one another denotes hydrogen or a group optionally substituted by one or more identical or different R^{h}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl; and
each R^{h} is selected independently of one another from among hydrogen, C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl, a tautomer thereof, a racemate thereof, an enantiomer thereof, a diastereomer thereof, or a mixture of any of the foregoing, or a pharmacologically acceptable salt thereof.

9. The compound according to claim 8, wherein R³ denotes hydrogen.

10. The compound according to claim 8, wherein R⁴ denotes hydrogen.

11. The compound according to claim 8, wherein R⁵ denotes C₁₋₃alkyl.

12. A pharmaceutical preparation comprising as active substance one or more compounds of formula (I) according to claim 8 and one or more conventional excipients or carriers.

13. The pharmaceutical preparation according to claim 12 comprising at least one other cytostatic or cytotoxic active substance different from any compounds according to claim 8.

14. A method for the prevention or treatment of cancer, infections, inflammatory diseases or autoimmune diseases which comprises administering a therapeutically effective amount of a compound according to claim 8.
